(19) United States
(12) Patent Application Publication
(10) Pub. No.: US 2026/0062706 A1
(43) Pub. Date: Mar. 5, 2026

(54) Treatment Of Pan-Cancer Phenotype With Regulator of Telomere Elongation Helicase 1 (RTEL1) Inhibitors

(71) Applicant: Regeneron Pharmaceuticals, Inc., Tarrytown, NY (US)

(72) Inventors: Michael Kessler, Tarrytown, NY (US); Eric Jorgenson, Tarrytown, NY (US); Adolfo Ferrando, Tarrytown, NY (US); Jing Tang, Tarrytown, NY (US); Silvia Alvarez, Tarrytown, NY (US); Kathryn Burch, Tarrytown, NY (US); Kuan-Han Wu, Tarrytown, NY (US)

(21) Appl. No.: 19/314,288

(22) Filed: Aug. 29, 2025

Related U.S. Application Data
(60) Provisional application No. 63/689,040, filed on Aug. 30, 2024.

(51) Int. Cl. C12N 15/113; C12Q 1/6886

(52) U.S. Cl. CPC C12N 15/1137; C12Q 1/6886; C12N 2310/11; C12N 2310/14; C12Q 2600/156

(57) ABSTRACT

The present disclosure generally relates to the treatment of subjects having pan-cancer phenotype or at risk of developing pan-cancer phenotype by administering a Regulator of Telomere Elongation Helicase 1 (RTEL1) inhibitor to the subject.

### FIELD

The present disclosure generally relates to the treatment of subjects having pan-cancer phenotype or at risk of developing pan-cancer phenotype, by administering a Regulator of Telomere Elongation Helicase 1 (RTEL1) inhibitor to the subject, and to methods of identifying subjects having an increased risk of developing pan-cancer phenotype.

### BACKGROUND

Cancers are typically classified by the location of origination in the body. Recently, however, cancer classification analysis has been focused on a molecular approach. Preliminary analyses has revealed that cancers that start in different organs may actually share similarities at the molecular level, whereas cancers that originate from the same tissue can have very different genomic profiles. Such an approach, termed the pan-cancer approach, involves a large analysis of genomic and molecular data characterizing numerous different types of cancer from tens of thousands of patients. Such analysis has resulted in a comprehensive molecular analysis of the complete set of TCGA tumor data. The results showed that, based on their cellular and genetic profile and independent of their anatomic site of origin, oncogenic signaling pathway(s), genomic instability, plasticity, and immune evasion, these numerous tumor types could be reclassified into a smaller subset of different molecular types, or “clusters” (Hanahan, Cancer Discov., 2022, 12, 31-46). Nearly two-thirds of these clusters were considered heterogeneous because they contained up to 25 different histological tumor types that, previously, would all be treated differently. These molecular analyses and clustering results, now also linked to multiple clinical outcome endpoints, are available to clinicians and researchers worldwide (TCGA Pan-Cancer Atlas; UCSC Tumor Map).

Regulator of Telomere Elongation Helicase 1 (RTEL1) is a DNA helicase that unwinds secondary structures (telomeric loops or G-quadruplex structures) to allow replication at difficult-to-replicate sites and suppress telomere fragility. RTEL1 facilitates DNA replication at telomeres (telomeric MiDAS) through T-loop disassembly to prevent catastrophic telomere processing by SLX4-SLX1 complex (Brenner et al., Trends in Biochem. Sci., 2022, 47, 506-517). Mutations in RTEL1 have been associated with inherited bone marrow failure syndromes (e.g., Dyskeratosis Congenita), Hoyeraal-Hreidarsson (HH) syndrome, a severe form of Dyskeratosis Congenita, as well as familial pulmonary fibrosis. Dyskeratosis congenita and Hoyeraal-Hreidarsson are complex telomere biology disorders, and are not exclusively driven by RTEL1 mutations. While these conditions are thought to be inherited in mendelian fashion, population scale analysis of RTEL1 genetic variants is lacking, and questions remain as to how common and rare variants in RTEL1 influence the phenotypic landscape. Somatic amplifications detected in nearly 30% of high-grade glioma, with copy-number changes correlating with RTEL1 expression, support a gain-of-function oncogenic role. Mouse models demonstrate that overexpression of RTEL1 is tumorigenic (Wu et al., Transgenic Res., 2012, 21, 1109-15). In addition, intronic variants in intron 12 (rs6010620) and intron 17 (rs4809324) of RTEL1 are associated with an increased risk of glioblastoma (Wrensch et al. Nat. Genet., 2009, 41, 905-908). Intronic RTEL1 splice region SNPs (rs41309931 and rs3787098) have been associated with increased risk of lung adenocarcinoma (Byun et al., Carcinogenesis, 2018, 39, 1135-1140; and Yan et al., Oncotarget, 2016, 7, 70475-70480).

### SUMMARY

The present disclosure provides methods of treating a subject having pan-cancer phenotype, or at risk of developing pan-cancer phenotype, the methods comprising administering an RTEL1 inhibitor to the subject.

The present disclosure also provides methods of treating a subject having pan-cancer phenotype or at risk of developing pan-cancer phenotype by administering a pan-cancer phenotype therapeutic agent, the methods comprising: determining or having determined whether the subject has an RTEL1 variant nucleic acid molecule, by: obtaining or having obtained a biological sample from the subject; and performing or having performed a sequence analysis on the biological sample to determine if the subject has a genotype comprising an RTEL1 variant nucleic acid molecule; and administering or continuing to administer the pan-cancer phenotype therapeutic agent in an amount that is the same as or less than a standard dosage amount, and/or administering an RTEL1 inhibitor to a subject that is RTEL1 reference; administering or continuing to administer the pan-cancer phenotype therapeutic agent in an amount that is the same as or less than a standard dosage amount, and/or administering an RTEL1 inhibitor to a subject that is heterozygous for the RTEL1 variant nucleic acid molecule; or administering or continuing to administer the pan-cancer phenotype therapeutic agent in a standard dosage amount to a subject that is homozygous for the RTEL1 variant nucleic acid molecule; wherein the presence the RTEL1 variant nucleic acid molecule indicates the subject has a decreased risk of developing pan-cancer phenotype.

The present disclosure also provides methods of identifying a subject having an increased risk of developing pan-cancer phenotype, the methods comprising: determining or having determined the presence or absence of an RTEL1 variant nucleic acid molecule in a biological sample obtained from the subject; wherein: when the subject is RTEL1 reference, then the subject has an increased risk of developing pan-cancer phenotype; and when the subject is heterozygous or homozygous for the RTEL1 variant nucleic acid molecule, then the subject has a decreased risk of developing pan-cancer phenotype.

The present disclosure also provides pan-cancer phenotype therapeutic agents for use in the treatment or prevention of pan-cancer phenotype in a subject having an RTEL1 variant nucleic acid molecule.

The present disclosure also provides RTEL1 inhibitors for use in the treatment or prevention of pan-cancer phenotype in a subject that is RTEL1 reference or is heterozygous for an RTEL1 variant nucleic acid molecule.

### BRIEF DESCRIPTION OF THE DRAWINGS

The accompanying figures, which are incorporated in and constitute a part of this specification, illustrate several features of the present disclosure and are intended to exemplify non-limiting embodiments of the present disclosure.

FIG. 1 shows results of studies indicating a pan cancer protective loss-of-function signal in the RTEL1 gene.

FIG. 2 shows results of studies indicating a protective RTEL1 signal is observed across cancer subtypes.

FIG. 3 shows results of studies indicating an RTEL1 loss-of-function signal is associated with malignant cancers.

FIG. 4 shows results of studies indicating that telomere length is significantly shorter in RTEL1 pLoF carriers.

FIG. 5 shows results of studies indicating that genetic determinants of longer telomeres are associated with an increased risk of cancer.

FIG. 6 shows results of studies indicating that the protective RTEL1 association signal can be observed in a longitudinal analysis of time to first cancer.

FIG. 7 shows age distrubutions by RTEL1 variant and carrier status.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by +10% and remain within the scope of the disclosed embodiments.

As used herein, the term “comprising” may be replaced with “consisting” or “consisting essentially of” in particular embodiments as desired.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horses, cows, and pigs), companion animals (such as, for example, dogs and cats), laboratory animals (such as, for example, mice, rats, and rabbits), and non-human primates. In some embodiments, the subject is a human. In some embodiments, the human is a patient under the care of a physician.

It has been observed in accordance with the present disclosure that rare and common RTEL1 variant nucleic acid molecules (whether these variants are homozygous or heterozygous in a particular subject) associate with a decreased risk of developing pan-cancer phenotype. In particular, whole-exome sequencing and electronic health record data from 947,128 individuals collected across 8 biobanks have been leveraged to identify common and rare RTEL1 variants for testing for association with 3031 quantitative and 2271 binary traits. Notably, it was discovered that rare (AAF≤0.001) putative loss-of-function (pLoF) variants are associated with protection from a pan-cancer phenotype when tested in a gene burden framework (OR=0.54, P=1.90×10⁻⁶). At the cancer subtype level, this protective signal is most pronounced for prostate cancer in men (OR=0.36, P=7.90×10⁻⁴), but remained nominally significant when testing for association with a pan-cancer phenotype in women only (OR=0.63, P=2.40×10⁻³). Survival analysis using longitudinal data from the UK Biobank provided additional support, with a longer time to first solid cancer observed in RTEL1 pLoF carriers compared to non-carriers (HR=0.69, P=0.1). While ploF carrier status was significantly associated with binary and quantitative traits indicative of telomeropathies, such as interstitial lung disease (OR=7.1, P=2.44×10⁻¹¹), significantly shorter telomeres (−0.827 normalized units, P=1.56×10⁻⁵⁰), and hematologic changes consistent with aplastic anemia (P<3×10⁻¹³), pLoF carriers had a normal age distribution and only 5/333 (1.5%) of them in the UK Biobank were diagnosed with a congenital disorder. It is believed that RTEL1 pLoF variant nucleic acid molecules have not been associated with pan-cancer phenotype in humans. These findings represent the first statistically significant human genetic-based protective pan-cancer association attributable to the loss-of-function of a critical component of telomeric maintenance, and represents a novel target for pan-cancer therapeutic development. Therefore, subjects that are RTEL1 reference or heterozygous for an RTEL1 variant nucleic acid molecule may be treated with an RTEL1 inhibitor such that pan-cancer phenotype is inhibited or prevented, the symptoms thereof are reduced or prevented, and/or development of symptoms is repressed or prevented. It is also believed that such subjects having pan-cancer phenotype may further be treated with one or more pan-cancer phenotype therapeutic agents that treats or inhibits pan-cancer phenotype. In addition, the present disclosure provides methods of leveraging the presence or absence of RTEL1 variant nucleic acid molecules in subjects to identify or stratify risk is such subjects of developing pan-cancer phenotype, or to diagnose subjects as having an increased risk of developing pan-cancer phenotype.

For purposes of the present disclosure, any particular subject, such as a human, can be categorized as having one of three RTEL1 genotypes: i) RTEL1 reference; ii) heterozygous for an RTEL1 variant nucleic acid molecule; or iii) homozygous for an RTEL1 variant nucleic acid molecule. A subject is RTEL1 reference when the subject does not have a copy of an RTEL1 variant nucleic acid molecule. A subject is heterozygous for an RTEL1 variant nucleic acid molecule when the subject has a single copy of an RTEL1 variant nucleic acid molecule. A subject is homozygous for an RTEL1 variant nucleic acid molecule when the subject has two copies of an RTEL1 variant nucleic acid molecule.

In any of the embodiments described herein, the RTEL1 variant nucleic acid molecule can be any nucleic acid molecule (such as, a genomic nucleic acid molecule, an mRNA molecule, or a cDNA molecule produced from an mRNA molecule) encoding an RTEL1 variant polypeptide having a partial loss-of-function, a complete loss-of-function, a predicted partial loss-of-function, or a predicted complete loss-of-function. A subject who has an RTEL1 polypeptide having a partial loss-of-function (or predicted partial loss-of-function) is hypomorphic for RTEL1. In some embodiments, the RTEL1 variant nucleic acid molecule results in decreased or aberrant expression or activity of RTEL1 mRNA or polypeptide. In some embodiments, the RTEL1 variant nucleic acid molecule is associated with a reduced in vitro response to RTEL1 ligands compared with reference RTEL1. In some embodiments, the RTEL1 variant nucleic acid molecule is a splice-site variant, a stop-gain variant, a start-loss variant, a stop-loss variant, a frameshift variant, an in-frame indel variant, or a variant that encodes a truncated RTEL1 variant polypeptide. In some embodiments, the RTEL1 variant nucleic acid molecule is a missense variant nucleic acid molecule. In some embodiments, the RTEL1 variant nucleic acid molecule comprises a single nucleotide polymorphism (SNP). In some embodiments, the RTEL1 variant nucleic acid molecule comprises a variation in a coding region. In some embodiments, the RTEL1 variant nucleic acid molecule does not comprise a variation in a non-coding region, except for a splice acceptor region (two bases before the start of any exon except the first). In some embodiments, the RTEL1 variant nucleic acid molecule results or is predicted to result in a premature truncation of an RTEL1 polypeptide compared to the reference RTEL1. In some embodiments, the RTEL1 variant nucleic acid molecule is a variant that is predicted to be damaging to the protein function (and hence, in this case, protective to the human) by in vitro prediction algorithms such as Polyphen, SIFT, or similar algorithms. In some embodiments, the RTEL1 variant nucleic acid molecule is a variant that causes or is predicted to cause a nonsynonymous amino acid substitution in an RTEL1 nucleic acid molecule and whose allele frequency is less than 1/100 alleles in the population from which the subject is selected. In some embodiments, the RTEL1 variant nucleic acid molecule is any rare missense variant (allele frequency <0.1%; or 1 in 1,000 alleles), or any splice-site, stop-gain, start-loss, stop-loss, frameshift, or in-frame indel, or other frameshift RTEL1 variant.

In any of the embodiments described herein, the RTEL1 variant genomic nucleic acid molecule may include one or more variations at any of the positions of chromosome 20 (i.e., positions 63,657,810-63,696,253) using the nucleotide sequence of the RTEL1 reference genomic nucleic acid molecule in the GRCh38/hg38 human genome assembly (see, ENSG00000258366.12, ENST00000360203 annotated in the Ensembl database (URL: world wide web at “useast.ensembl.org/Homo_sapiens/Gene/Summary?db=core; g=ENSG00000258366;r=20:63657810-63696253”)) as a reference sequence. The sequences provided in these transcripts for the RTEL1 genomic nucleic acid molecule are only exemplary sequences. Other sequences for the RTEL1 genomic nucleic acid molecule are also possible.

In any of the embodiments described herein, the RTEL1 variant nucleic acid molecule may comprise any one or more of the genetic variations in the genomic nucleic acid molecule (referring to the chromosome: positions set forth in the GRCh38/hg38 human genome assembly) listed in Table 1, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63687647:T:G, 20:63687688:C:G, 20:63693211:C:T, 20:63685789:A:C, 20:63662841:C:T, 20:63695551:CTG:C, 20:63667489:C:T, 20:63694876:C:T, 20:63661341:C:T, 20:63688315:G:A, 20:63662559:G:C, 20:63690137:A:C, 20:63667492:G:A, 20:63678268:T:C, 20:63690835:G:T, 20:63694489:G:A, 20:63687706:C:T, and 20:63667554:G:T, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63667554:G:T, 20:63685789:A:C, 20:63693211:C:T (rs398123017), 20:63694489:G:A, and 20:63695551:CTG:C (rs1316931773), or an mRNA molecule produced therefrom, or a cDNA molecule produced from the mRNA molecule.

TABLE 1

| Variant | Type | rsID |
|---|---|---|
| 20:63659463:C:T | stop_gained | |
| 20:63659466:C:T | stop_gained | |
| 20:63659472:T:TTGGC | frameshift | |
| 20:63659499:C:T | stop_gained | |
| 20:63659505:G:A | splice_donor | |
| 20:63661371:G:A | stop_gained | |
| 20:63661373:C:T | stop_gained | rs1457184127 |
| 20:63661385:C:T | stop_gained | rs368311594 |
| 20:63661394:ATC:A | frameshift | |
| 20:63661418:AG:A | frameshift | |
| 20:63661433:CT:C | frameshift | |
| 20:63661450:C:CT | frameshift | |
| 20:63661895:AC:A | frameshift | |
| 20:63661908:AC:A | frameshift | |
| 20:63661941:C:CG | frameshift | |
| 20:63661944:G:C | splice_donor | |
| 20:63662607:CAA:C | frameshift | rs773025155 |
| 20:63662876:C:A | stop_gained | rs1161373315 |
| 20:63662890:G:A | splice_donor | rs745549355 |
| 20:63666003:GA:G | frameshift | |
| 20:63666065:C:CGGAA | frameshift | |
| 20:63666080:G:A | splice_donor | |
| 20:63666081:T:G | splice_donor | |
| 20:63667467:A:G | splice_acceptor | rs2090159848 |
| 20:63667554:G:T | splice_donor | |
| 20:63667554:G:A | splice_donor | |
| 20:63667555:T:C | splice_donor | |
| 20:63672622:G:C | splice_donor | |
| 20:63673937:TAGG:T | splice_acceptor | |
| 20:63673938:A:G | splice_acceptor | rs745431021 |
| 20:63674045:C:T | stop_gained | |
| 20:63674070:TC:T | frameshift | |
| 20:63678144:G:C | splice_acceptor | |
| 20:63678179:GAA:G | frameshift | |
| 20:63678347:G:T | splice_donor | rs895722334 |
| 20:63679894:C:CA | frameshift | |
| 20:63679914:C:A | stop_gained | |
| 20:63679929:T:TC | frameshift | |
| 20:63679947:G:A | splice_donor | |
| 20:63680717:C:T | stop_gained | |
| 20:63680721:T:C | splice_donor | |
| 20:63685574:C:CT | frameshift | |
| 20:63685586:C:T | stop_gained | |
| 20:63685598:G:A | splice_donor | |
| 20:63685789:A:C | splice_acceptor | |
| 20:63685869:C:T | stop_gained | rs2090581105 |
| 20:63687636:A:G | splice_acceptor | |
| 20:63687637:G:A | splice_acceptor | |
| 20:63687771:G:A | splice_donor | rs1224194611 |
| 20:63687933:TCAG:T | splice_acceptor | |
| 20:63687935:A:C | splice_acceptor | |
| 20:63687936:G:A | splice_acceptor | rs863225129 |
| 20:63687944:CCA:C | frameshift | |
| 20:63687960:AC:A | frameshift | |
| 20:63688139:G:GTT | frameshift | |
| 20:63688146:G:T | stop_gained | |
| 20:63688165:TG:T | frameshift | |
| 20:63688320:GC:G | frameshift | |
| 20:63688383:G:A | stop_gained | |
| 20:63688526:A:G | splice_acceptor | |
| 20:63688527:G:A | splice_acceptor | |
| 20:63689065:CT:C | frameshift | |
| 20:63689531:GA:G | frameshift | rs2090675337 |
| 20:63689566:A:AC | frameshift | |
| 20:63689567:C:A | stop_gained | |
| 20:63689581:A:AC | frameshift | rs1555811742 |
| 20:63689597:CAA:C | frameshift | |
| 20:63689628:C:T | stop_gained | |
| 20:63689639:T:TG | frameshift | rs1263776141 |
| 20:63689649:G:A | splice_donor | |
| 20:63689749:G:T | splice_acceptor | |
| 20:63689802:AG:A | frameshift | |
| 20:63689809:CG:C | frameshift | |
| 20:63689813:C:T | stop_gained | rs768188490 |
| 20:63689820:TC:T | frameshift | rs2090684427 |
| 20:63689839:C:CT | frameshift | |
| 20:63689866:G:T | splice_donor | rs1281899571 |
| 20:63690085:A:G | splice_acceptor | rs2090696292 |
| 20:63690120:“CTCCTGGGT” | frameshift | |
| followed by “GCG”:C | | |
| 20:63690172:C:T | stop_gained | |
| 20:63690191:TC:T | frameshift | rs2090699833 |
| 20:63690205:C:T | stop_gained | rs377024903 |
| 20:63690211:G:A | splice_donor | |
| 20:63690312:C:CG | frameshift | |
| 20:63690336:G:T | stop_gained | |
| 20:63690339:G:T | stop_gained | |
| 20:63690396:AAG:A | frameshift | |
| 20:63690429:C:T | stop_gained | |
| 20:63690442:G:C | splice_donor | rs 776744306 |
| 20:63690443:T:C | splice_donor | |
| 20:63690809:AC:A | frameshift | |
| 20:63690855:C:T | stop_gained | |
| 20:63690855:CA:C | frameshift | |
| 20:63690875:GC:G | frameshift | |
| 20:63690884:CA:C | frameshift | |
| 20:63690925:C:CG | frameshift | |
| 20:63690945:C:T | stop_gained | |
| 20:63691741:G:T | splice_acceptor | |
| 20:63691759:CCT:C | frameshift | |
| 20:63691768:CCTGT:C | frameshift | rs752833281 |
| 20:63691778:A:T | stop_gained | |
| 20:63691788:C:CA | frameshift | |
| 20:63691799:C:T | stop_gained | rs961593162 |
| 20:63692803:A:T | splice_acceptor | |
| 20:63692826:C:T | stop_gained | |
| 20:63692868:G:T | stop_gained | |
| 20:63692874:AG:A | frameshift | |
| 20:63692877:C:T | stop_gained | rs866637461 |
| 20:63692884:AC:A | frameshift | |
| 20:63692901:CA:C | frameshift | |
| 20:63692920:A:AG | frameshift | |
| 20:63692934:TTC:T | frameshift | |
| 20:63692940:GC:G | frameshift | |
| 20:63692954:T:A | stop_gained | |
| 20:63692954:T:TC | frameshift | |
| 20:63692959:GC:G | frameshift | rs1449687529 |
| 20:63693004:G:T | splice_donor | rs1421904176 |
| 20:63693142:G:A | splice_acceptor | |
| 20:63693144:C:CTTCT | frameshift | |
| 20:63693175:C:T | stop_gained | |
| 20:63693187:G:T | stop_gained | |
| 20:63693199:C:T | stop_gained | |
| 20:63693211:C:T | stop_gained | rs398123017 |
| 20:63693223:T:TATCG | frameshift | rs1285773934 |
| 20:63693243:TC:T | frameshift | rs1285014916 |
| 20:63693247:C:T | stop_gained | rs373740199 |
| 20:63693253:C:T | stop_gained | |
| 20:63693262:C:T | stop_gained | rs560165598 |
| 20:63693284:G:T | splice_donor | |
| 20:63693284:G:C | splice_donor | rs1370553261 |
| 20:63694396:TG:T | frameshift | |
| 20:63694419:C:T | stop_gained | rs 747743374 |
| 20:63694422:C:T | stop_gained | |
| 20:63694428:G:GA | frameshift | |
| 20:63694457:G:GC | frameshift | |
| 20:63694468:C:CG | frameshift | |
| 20:63694489:G:A | splice_donor | |
| 20:63694490:T:C | splice_donor | |
| 20:63694739:A:T | splice_acceptor | rs377461417 |
| 20:63694739:A:C | splice_acceptor | rs377461417 |
| 20:63694765:G:A | stop_gained | |
| 20:63694800:C:T | stop_gained | rs2090929102 |
| 20:63694863:C:T | stop_gained | |
| 20:63694943:T:TG | frameshift | |
| 20:63694960:TC:T | frameshift | |
| 20:63694975:G:A | splice_donor | |
| 20:63694976:C:T | splice_donor | |
| 20:63695110:C:T | stop_gained | |
| 20:63695223:T:C | splice_donor | |
| 20:63695223:T:G | splice_donor | |
| 20:63695327:G:A | splice_acceptor | rs1205986855 |
| 20:63695334:C:CA | frameshift | |
| 20:63695369:ATC:A | frameshift | |
| 20:63695379:TC:T | frameshift | rs1363124795 |
| 20:63695383:TAGAC:T | frameshift | rs1238498783 |
| 20:63695383:T:TA | frameshift | |
| 20:63695387:C:T | stop_gained | rs776525427 |
| 20:63695387:CAG:C | frameshift | |
| 20:63695470:ATC:A | frameshift | |
| 20:63695483:G:T | stop_gained | |
| 20:63695510:C:T | stop_gained | |
| 20:63695539:CCT:C | frameshift | rs1363658406 |
| 20:63695551:CTG:C | frameshift | rs1316931773 |
| 20:63695551:CTGTG:C | frameshift | |
| 20:63695551:CTG:C | frameshift | rs1316931773 |
| 20:63695583:TG:T | frameshift | |
| 20:63695584:“GGTGCCCTT” | frameshift | |
| followed by “CCA”:G | | |
| 20:63695587:GC:G | frameshift | rs1054506840 |
| 20:63695589:CCT:C | frameshift | rs1162803306 |
| 20:63695602:TGC:T | frameshift | |
| 20:63695635:G:A | stop_gained | |
| 20:63695780:CT:C | frameshift | |
| 20:63695784:AG:A | frameshift | |
| 20:63695832:C:T | stop_gained | |
| 20:63695842:G:A | stop_gained | |
| 20:63695843:G:A | stop_gained | |
| 20:63695856:TGA:T | frameshift | |

For subjects that are genotyped or determined to be RTEL1 reference, such subjects have an increased risk of developing pan-cancer phenotype. For subjects that are genotyped or determined to be either RTEL1 reference or heterozygous for an RTEL1 variant nucleic acid molecule, such subjects can be treated with an RTEL1 inhibitor.

In any of the embodiments described herein, the subject in whom pan-cancer phenotype is prevented by administering an RTEL1 inhibitor may be anyone at risk for developing pan-cancer phenotype including, but not limited to, subjects with a genetic predisposition for developing pan-cancer phenotype. In some embodiments, administering an RTEL1 inhibitor to a subject having pan-cancer phenotype may be carried out to prevent development of another occurrence of pan-cancer phenotype in a subject who has already had pan-cancer phenotype. In any of the embodiments described herein, the methods can be used to improve pan-cancer phenotype.

In any of the embodiments described herein, the RTEL1 predicted loss-of-function polypeptide can be any RTEL1 polypeptide having a partial loss-of-function, a complete loss-of-function, a predicted partial loss-of-function, or a predicted complete loss-of-function. Any one or more (i.e., any combination) of the RTEL1 variant nucleic acid molecules described herein can be used within any of the methods described herein to determine whether a subject has an increased or decreased risk of developing pan-cancer phenotype. The combinations of particular variants can form a mask used for statistical analysis of the particular correlation of RTEL1 and an increased or decreased risk of developing pan-cancer phenotype. In some embodiments, the mask used for statistical analysis of the particular correlation of RTEL1 and an increased or decreased risk of developing pan-cancer phenotype can exclude any one or more of these RTEL1 variant nucleic acid molecules described herein.

In any of the embodiments described herein, the subject can have pan-cancer phenotype. In any of the embodiments described herein, the subject can be at risk of developing pan-cancer phenotype. In any of the embodiments described herein, the pan-cancer phenotype is a malignant cancer. In any of the embodiments described herein, the pan-cancer phenotype is a solid cancer. In any of the embodiments described herein, the pan-cancer phenotype is chosen from the following cancers: adrenal, bladder, bone, cartilage, brain, breast, cervical, colon, esophagus, kidney, lip, oral cavity, pharynx, liver, lung, melanoma, ovarian, pancreatic, prostate, rectal, stomach, testicular, thyroid, and blood. In any of the embodiments described herein, the pan-cancer phenotype is chosen from the following cancers: breast, colon, kidney, melanoma, prostate, and blood. In any of the embodiments described herein, the pan-cancer phenotype is not a non-melanoma skin cancer.

The present disclosure provides methods of treating a subject having pan-cancer phenotype or at risk of developing pan-cancer phenotype, the methods comprising administering an RTEL1 inhibitor to the subject.

In some embodiments, the RTEL1 inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of an RTEL1 nucleic acid molecule. In some embodiments, the antisense RNA, siRNA, or shRNA hybridizes to a sequence within an RTEL1 genomic nucleic acid molecule or mRNA molecule and decreases expression of the RTEL1 polypeptide in a cell in the subject. In some embodiments, the RTEL1 inhibitor comprises an antisense molecule that hybridizes to an RTEL1 genomic nucleic acid molecule or mRNA molecule and decreases expression of the RTEL1 polypeptide in a cell in the subject. In some embodiments, the RTEL1 inhibitor comprises an siRNA that hybridizes to an RTEL1 genomic nucleic acid molecule or mRNA molecule and decreases expression of the RTEL1 polypeptide in a cell in the subject. In some embodiments, the RTEL1 inhibitor comprises an shRNA that hybridizes to an RTEL1 genomic nucleic acid molecule or mRNA molecule and decreases expression of the RTEL1 polypeptide in a cell in the subject.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂),CH₃, —O(CH₂)ₙ-ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

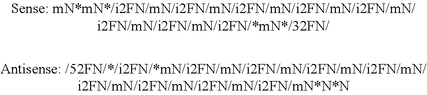

wherein: “N” is the base; “2F” is a 2′-F modification; “m” is a 2′-O-methyl modification, “I” is an internal base; and “*” is a phosphorothioate backbone linkage.

In any of the embodiments described herein, the inhibitory nucleic acid molecules may be administered, for example, as one to two hour i.v. infusions or s.c. injections. In any of the embodiments described herein, the inhibitory nucleic acid molecules may be administered at dose levels that range from about 50 mg to about 900 mg, from about 100 mg to about 800 mg, from about 150 mg to about 700 mg, or from about 175 to about 640 mg (2.5 to 9.14 mg/kg; 92.5 to 338 mg/m²-based on an assumption of a body weight of 70 kg and a conversion of mg/kg to mg/m² dose levels based on a mg/kg dose multiplier value of 37 for humans).

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly (lactic acid) (PLA) microspheres, poly (D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the RTEL1 inhibitor comprises a nuclease agent that induces one or more nicks or double-strand breaks at a recognition sequence(s) or a DNA-binding protein that binds to a recognition sequence within an RTEL1 genomic nucleic acid molecule. The recognition sequence can be located within a coding region of the RTEL1 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the RTEL1 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify an RTEL1 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of RTEL1 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in an RTEL1 genomic nucleic acid molecule or it can be a nickase that creates a single-strand break in an RTEL1 genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. In some embodiments, a Cas system, such as Cas12a, can have multiple gRNAs encoded into a single crRNA. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of RTEL1 genomic nucleic acid molecules can be generated by contacting a cell with a Cas protein and one or more gRNAs that hybridize to one or more gRNA recognition sequences within a target genomic locus in the RTEL1 genomic nucleic acid molecule. The gRNA recognition sequence can include or be proximate to the start codon of an RTEL1 genomic nucleic acid molecule or the stop codon of an RTEL1 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in an RTEL1 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where Nis any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within an RTEL1 genomic nucleic acid molecule. An exemplary gRNA is a gRNA effective to direct a Cas enzyme to bind to or cleave an RTEL1 genomic nucleic acid molecule, wherein the gRNA comprises a DNA-targeting segment that hybridizes to a gRNA recognition sequence within the RTEL1 genomic nucleic acid molecule. Exemplary gRNAs comprise a DNA-targeting segment that hybridizes to a gRNA recognition sequence present within an RTEL1 genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

The Cas protein and the gRNA form a complex, and the Cas protein cleaves the RTEL1 genomic nucleic acid molecule. The Cas protein can cleave the nucleic acid molecule at a site within or outside of the nucleic acid sequence present in the RTEL1 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the RTEL1 genomic nucleic acid molecule to which a DNA-targeting segment of a gRNA will bind.

Such methods can result, for example, in an RTEL1 genomic nucleic acid molecule in which a region of the RTEL1 genomic nucleic acid molecule is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the RTEL1 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In any of the methods of treatment or prevention described herein, the subject being treated may comprise an RTEL1 variant nucleic acid molecule. In some embodiments, the subject being treated is heterozygous for the RTEL1 variant nucleic acid molecule. In some embodiments, the subject being treated is homozygous for the RTEL1 variant nucleic acid molecule. In some embodiments, the subject being treated is RTEL1 reference. The RTEL1 variant nucleic acid molecule can be any of the RTEL1 variant nucleic acid molecules disclosed herein. In some embodiments, the RTEL1 variant nucleic acid molecule is a RTEL1 variant genomic nucleic acid molecule that comprises the genetic variation listed in Table 1, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63687647:T:G, 20:63687688:C:G, 20:63693211:C:T, 20:63685789:A:C, 20:63662841:C:T, 20:63695551:CTG:C, 20:63667489:C:T, 20:63694876:C:T, 20:63661341:C:T, 20:63688315:G:A, 20:63662559:G:C, 20:63690137:A:C, 20:63667492:G:A, 20:63678268:T:C, 20:63690835:G:T, 20:63694489:G:A, 20:63687706:C:T, and 20:63667554:G:T, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63667554:G:T, 20:63685789:A:C, 20:63693211:C:T (rs398123017), 20:63694489:G:A, and 20:63695551:CTG:C (rs1316931773), or an mRNA molecule produced therefrom, or a cDNA molecule produced from the mRNA molecule.

In some embodiments, the methods of treatment or prevention further comprise detecting the presence or absence of an RTEL1 variant nucleic acid molecule in a biological sample from the subject. In some embodiments, the RTEL1 variant nucleic acid molecule can be any of the RTEL1 variant nucleic acid molecules disclosed herein. In some embodiments, the RTEL1 variant nucleic acid molecule is a RTEL1 variant genomic nucleic acid molecule that comprises the genetic variation listed in Table 1, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63687647:T: G, 20:63687688:C:G, 20:63693211:C:T, 20:63685789:A:C, 20:63662841:C:T, 20:63695551:CTG:C, 20:63667489:C:T, 20:63694876:C:T, 20:63661341:C:T, 20:63688315:G:A, 20:63662559:G:C, 20:63690137:A:C, 20:63667492:G:A, 20:63678268:T:C, 20:63690835:G:T, 20:63694489:G:A, 20:63687706:C:T, and 20:63667554:G:T, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63667554:G:T, 20:63685789:A:C, 20:63693211:C:T (rs398123017), 20:63694489:G:A, and 20:63695551:CTG:C (rs1316931773), or an mRNA molecule produced therefrom, or a cDNA molecule produced from the mRNA molecule.

The present disclosure also provides methods of treating a subject with a pan-cancer phenotype therapeutic agent that treats or inhibits pan-cancer phenotype, wherein the subject has pan-cancer phenotype or is at risk of developing pan-cancer phenotype. The methods comprise determining whether the subject has an RTEL1 variant nucleic acid molecule by obtaining or having obtained a biological sample from the subject, and performing or having performed a sequence analysis on the biological sample to determine if the subject has a genotype comprising the RTEL1 variant nucleic acid molecule. In embodiments where the subject is RTEL1 reference, the methods further comprise administering or continuing to administer the pan-cancer phenotype therapeutic agent in an amount that is the same as or less than a standard dosage amount to the subject, and/or administering an RTEL1 inhibitor to the subject. In embodiments where the subject is heterozygous for the RTEL1 variant nucleic acid molecule, the methods further comprise administering or continuing to administer the pan-cancer phenotype therapeutic agent in an amount that is the same as or less than a standard dosage amount to the subject, and/or administering an RTEL1 inhibitor to the subject. In embodiments where the subject is homozygous for the RTEL1 variant nucleic acid molecule, the methods further comprise administering or continuing to administer the pan-cancer phenotype therapeutic agent in a standard dosage amount to the subject. The presence of an RTEL1 variant nucleic acid molecule indicates the subject has a decreased risk of developing pan-cancer phenotype. In some embodiments, the subject is RTEL1 reference. In some embodiments, the subject is heterozygous for an RTEL1 variant nucleic acid molecule. In some embodiments, the subject is homozygous for an RTEL1 variant nucleic acid molecule. In any of the embodiments described herein, the RTEL1 inhibitor is an example of a pan-cancer phenotype therapeutic agent. In some embodiments, the RTEL1 variant nucleic acid molecule is a RTEL1 variant genomic nucleic acid molecule that comprises the genetic variation listed in Table 1, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63687647:T:G, 20:63687688:C:G, 20:63693211:C:T, 20:63685789:A:C, 20:63662841:C:T, 20:63695551:CTG:C, 20:63667489:C:T, 20:63694876:C:T, 20:63661341:C:T, 20:63688315:G:A, 20:63662559:G:C, 20:63690137:A:C, 20:63667492:G:A, 20:63678268:T:C, 20:63690835:G:T, 20:63694489:G:A, 20:63687706:C:T, and 20:63667554:G:T, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63667554:G:T, 20:63685789:A:C, 20:63693211:C:T (rs398123017), 20:63694489:G:A, and 20:63695551:CTG:C (rs1316931773), or an mRNA molecule produced therefrom, or a cDNA molecule produced from the mRNA molecule.

For subjects that are genotyped or determined to be either RTEL1 reference or heterozygous for an RTEL1 variant nucleic acid molecule, such subjects can be administered an RTEL1 inhibitor, as described herein.

Detecting the presence or absence of an RTEL1 variant nucleic acid molecule in a biological sample from a subject and/or determining whether a subject has an RTEL1 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when the subject is RTEL1 reference, the subject is administered a pan-cancer phenotype therapeutic agent in an amount that is the same as or less than a standard dosage amount, and/or a RTEL1 inhibitor. In some embodiments, when the subject is heterozygous for an RTEL1 variant nucleic acid molecule, the subject is administered a pan-cancer phenotype therapeutic agent in an amount that is the same as or less than a standard dosage amount, and/or a RTEL1 inhibitor.

In some embodiments, the treatment or prevention methods comprise detecting the presence or absence of a decrease in the expression of an RTEL1 variant mRNA or polypeptide in a biological sample from the subject. In some embodiments, when the subject does not have a decrease in the expression of an RTEL1 variant mRNA or polypeptide, the subject is administered a pan-cancer phenotype therapeutic agent in an amount that is the same as or less than a standard dosage amount, and/or an RTEL1 inhibitor. In some embodiments, when the subject has a decrease in the expression of an RTEL1 variant mRNA or polypeptide, the subject is administered a pan-cancer phenotype therapeutic agent in a standard dosage amount.

The present disclosure also provides methods of treating a subject with a pan-cancer phenotype therapeutic agent that treats or inhibits pan-cancer phenotype, wherein the subject has pan-cancer phenotype or is at risk of developing pan-cancer phenotype. The methods comprise determining whether the subject has a decrease in the expression of an RTEL1 variant mRNA or polypeptide by obtaining or having obtained a biological sample from the subject, and performing or having performed an assay on the biological sample to determine if the subject a decrease in the expression of an RTEL1 variant mRNA or polypeptide. In embodiments where the subject does not have a decrease in the expression of an RTEL1 variant mRNA or polypeptide, the methods further comprise administering or continuing to administer the pan-cancer phenotype therapeutic agent in an amount that is the same as or less than a standard dosage amount to the subject, and/or administering an RTEL1 inhibitor to the subject. In embodiments where the subject has a decrease in the expression of an RTEL1 variant mRNA or polypeptide, the methods further comprise administering or continuing to administer the pan-cancer phenotype therapeutic agent in a standard dosage amount to the subject. The presence of a decrease in the expression of an RTEL1 variant mRNA or polypeptide indicates the subject has a decreased risk of developing pan-cancer phenotype. In some embodiments, the subject has a decrease in the expression of an RTEL1 variant mRNA or polypeptide. In some embodiments, the subject does not have a decrease in the expression of an RTEL1 variant mRNA or polypeptide. In any of the embodiments described herein, the RTEL1 inhibitor is an example of a pan-cancer phenotype therapeutic agent. In some embodiments, the RTEL1 variant nucleic acid molecule is a RTEL1 variant genomic nucleic acid molecule that comprises the genetic variation listed in Table 1, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63687647:T:G, 20:63687688:C:G, 20:63693211:C:T, 20:63685789:A:C, 20:63662841:C:T, 20:63695551:CTG:C, 20:63667489:C:T, 20:63694876:C:T, 20:63661341:C:T, 20:63688315:G:A, 20:63662559:G:C, 20:63690137:A:C, 20:63667492:G:A, 20:63678268:T:C, 20:63690835:G:T, 20:63694489:G:A, 20:63687706:C:T, and 20:63667554:G:T, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63667554:G:T, 20:63685789:A:C, 20:63693211:C:T (rs398123017), 20:63694489:G:A, and 20:63695551:CTG:C (rs1316931773), or an mRNA molecule produced therefrom, or a cDNA molecule produced from the mRNA molecule.

Detecting a decrease in the expression of an RTEL1 variant mRNA or polypeptide can be carried out by a variety of known methods. In some embodiments, these methods can be carried out in vitro. In some embodiments, these methods can be carried out in situ. In some embodiments, these methods can be carried out in vivo. In any of these embodiments, the mRNA or polypeptide can be present within a cell obtained from the subject.

In some embodiments, the treatment or prevention methods comprise detecting the presence or absence of an RTEL1 variant polypeptide in a biological sample from the subject. In some embodiments, when the subject does not have an RTEL1 variant polypeptide, the subject is administered a pan-cancer phenotype therapeutic agent in an amount that is the same as or less than a standard dosage amount, and/or an RTEL1 inhibitor. In some embodiments, when the subject has an RTEL1 variant polypeptide, the subject is administered a pan-cancer phenotype therapeutic agent in standard dosage amount.

The present disclosure also provides methods of treating a subject with a pan-cancer phenotype therapeutic agent that treats or inhibits pan-cancer phenotype, wherein the subject has pan-cancer phenotype or is at risk of developing pan-cancer phenotype. The methods comprise determining whether the subject has an RTEL1 variant polypeptide by obtaining or having obtained a biological sample from the subject and performing or having performed an assay on the biological sample to determine if the subject has an RTEL1 variant polypeptide. When the subject does not have an RTEL1 variant polypeptide, the subject is administered the pan-cancer phenotype therapeutic agent in an amount that is the same as or less than a standard dosage amount, and/or an RTEL1 inhibitor. When the subject has an RTEL1 variant polypeptide, the subject is administered the pan-cancer phenotype therapeutic agent in a standard dosage amount. The presence of an RTEL1 variant polypeptide indicates the subject has a decreased risk of developing pan-cancer phenotype. In some embodiments, the subject has an RTEL1 variant polypeptide. In some embodiments, the subject does not have an RTEL1 variant polypeptide.

The present disclosure also provides methods of preventing a subject from developing pan-cancer phenotype by administering a pan-cancer phenotype therapeutic agent that prevents pan-cancer phenotype. In some embodiments, the method comprises determining whether the subject has an RTEL1 variant polypeptide by obtaining or having obtained a biological sample from the subject and performing or having performed an assay on the biological sample to determine if the subject has an RTEL1 variant polypeptide. When the subject does not have an RTEL1 variant polypeptide, the subject is administered the pan-cancer phenotype therapeutic agent in an amount that is the same as or less than a standard dosage amount, and/or an RTEL1 inhibitor. When the subject has an RTEL1 variant polypeptide, the subject is administered the pan-cancer phenotype therapeutic agent in a standard dosage amount. The presence of an RTEL1 variant polypeptide indicates the subject has a decreased risk of developing pan-cancer phenotype. In some embodiments, the subject has an RTEL1 variant polypeptide. In some embodiments, the subject does not have an RTEL1 variant polypeptide.

Detecting the presence or absence of an RTEL1 variant polypeptide in a biological sample from a subject and/or determining whether a subject has an RTEL1 variant polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the RTEL1 inhibitor is a small molecule. In some embodiments, the small molecule is low molecular weight (<900 daltons) organic compound.

In some embodiments, the RTEL1 inhibitor comprises an antibody, or antigen-binding fragment thereof. In some embodiments, the antibody, or antigen-binding fragment thereof, binds specifically to human RTEL1. In some embodiments, the antibody is a fully human monoclonal antibody (mAb), or antigen-binding fragment thereof, that specifically binds and neutralizes, inhibits, blocks, abrogates, reduces, or interferes with, at least one activity of RTEL1, in particular, human RTEL1. In some embodiments, an antibody or fragment thereof can neutralize, inhibit, block, abrogate, reduce, or interfere with, an activity of RTEL1 by binding to an epitope of RTEL1 that is directly involved in the targeted activity of RTEL1. In some embodiments, an antibody or fragment thereof can neutralize, inhibit, block, abrogate, reduce, or interfere with, an activity of RTEL1 by binding to an epitope of RTEL1 that is not directly involved in the targeted activity of RTEL1, but the antibody or fragment binding thereto sterically or conformationally inhibits, blocks, abrogates, reduces, or interferes with, the targeted activity of RTEL1. In some embodiments, an antibody or fragment thereof binds to an epitope of RTEL1 that is not directly involved in the targeted activity of RTEL1 (i.e., a non-blocking antibody), but the antibody or fragment binding thereto results in the enhancement of the clearance of RTEL1 from the circulation, compared to the clearance of RTEL1 in the absence of the antibody or fragment thereof, thereby indirectly inhibiting, blocking, abrogating, reducing, or interfering with, an activity of RTEL1. Clearance of RTEL1 from the circulation can be particularly enhanced by combining two or more different non-blocking antibodies that do not compete with one another for specific binding to RTEL1. The antibodies can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy et al., J. Immunol., 2000, 164, 1925-1933).

In some embodiments, the antibody or antigen-binding fragment thereof specifically binds to RTEL1 with an equilibrium dissociation constant (K_{D}) of about 7 nM or less, about 6 nM or less, about 5 nM or less, about 4 nM or less, about 3 nM or less, about 2 nM or less, or about 1 nM or less, as measured by surface plasmon resonance assay (for example, BIACORE™). In some embodiments, the antibody exhibits a K_{D} of about 800 pM or less, about 700 pM or less; about 600 pM or less; about 500 pM or less; about 400 pM or less; about 300 pM or less; about 200 pM or less; about 100 pM or less; or about 50 pM or less.

In some embodiments, the anti-RTEL1 antibodies have a modified glycosylation pattern. In some applications, modification to remove undesirable glycosylation sites may be useful, or e.g., removal of a fucose moiety to increase antibody dependent cellular cytotoxicity (ADCC) function (see, Shield et al., J. Biol. Chem., 2002, 277, 26733). In other applications, removal of N-glycosylation site may reduce undesirable immune reactions against the therapeutic antibodies or increase affinities of the antibodies. In yet other applications, modification of galactosylation can be made in order to modify complement dependent cytotoxicity (CDC).

The present disclosure also provides compositions comprising a combination of an antibody or antigen-binding fragment thereof and a pan-cancer phenotype therapeutic agent.

In some embodiments, the pan-cancer phenotype therapeutic agents include, but are not limited to, immunotherapeutic agents and chemotherapeutic agents. Additional pan-cancer phenotype therapies include any therapy used to reduce or manage pan-cancer phenotype risk factors. In some embodiments, the pan-cancer phenotype therapeutic agent can be combined with an RTEL1 inhibitor. In some embodiments, a treatment therapy for pan-cancer phenotype is radiotherapy or tumor surgery. These treatment therapies may be delayed or avoided altogether by treatment with an RTEL1 inhibitor as described herein.

Examples of chemotherapeutic agents include, but are not limited to, methotrexate, taxol, mercaptopurine, thioguanine, hydroxyurea, cytarabine, mitomycin, cyclophosphamide, ifosfamide, nitrosourea, cisplatin, carboplatin, oxaliplatin, nedaplatin, triplatin tetranitrate, phenanthriplatin, picoplatin, satraplatin, dacarbazine, procarbizine, an etoposide, a campathecin, bleomycin, doxorubicin, idarubicin, daunorubicin, dactinomycin, distamycin A, etidium, netropsin, auristatin, amsacrine, prodigiosin, bortexomib, pibenzimol, tomaymycin, duocarmycin SA, plicamycin, mitoxantrone, asparaginase, vinblastine, vincristine, MG132, tunicamycin, oligomycin, vinorelbine, paclitaxel, docetaxel, CPT-11, gleevec, erlotinib, gefitinib, ibrutinib, crizotinib, ceritinib, flavopiridol, gemcitabine, lapatinib, navitoclax, sorafenib, regorafenib, ganetespib, irinotecan, or 5-fluorouracil, abraxane, actinomycin, alitretinoin, all-trans retinoic acid, altretamine, azacitidine, azathioprine, belotecan, bendamustine, bexarotene, bortezomib, busulfan, cabazitaxel, camptothecin, carboquone, carmustine, capecitabine, chlorambucil, chlormethine, chlorozotocin, doxifluridine, epirubicin, epothilone, exatecan, fotemustine, gimatecan, ixabepilone, larotaxel, lomustine, melphalan, melphalan flufenamide, mitobronitol, mitomycin C, nimustine, pemetrexed, pipobroman, ranimustine, romidepsin, semustine, streptozotocin, tafluposide, taxotere, temozolomide, tesetaxel, teniposide, thiotepa, tioguanine, topotecan, treosulfan, tretinoin, triaziquone, triethylenemelamine, valrubicin, vemurafenib, vindesine, vismodegib, vorinostat, or any combination thereof. In some embodiments, the chemotherapeutic agent is a combination of agents, such as, for example, methotrexate/vincristine/doxorubicin/cisplatin (MVAC) or gemcitabine/cisplatin.

Examples of immunotherapeutic agents include, but are not limited to, nivolumab, pembrolizumab, atezolizumab, durvalab, ipilumumab, avelumab, cetuxumab, bevacizumab, trastuzumab, cemiplimab, teclistamab, blinatumomab, gemtuzumab ozogamicin, brentuximab vedotin, trastuzumab emtansine, inotuzumab ozogamicin, polatuzumab vedotin, enfortumab vedotin, trastuzumab deruxtecan, sacituzumab govitecan, belantamab mafodotin, belantamab mafodotin, belantamab mafodotin, or any combination thereof.

Additional pan-cancer phenotype therapeutic agents include, but are not limited to, chaperones (i.e., proteins that help to fold other proteins correctly; chaperones may be used to help to stabilize RTEL1 and prevent it from being damaged); DNA repair enzymes (i.e., enzymes that can repair damaged DNA; DNA repair enzymes may be used to repair the DNA damage that is caused by mutations in RTEL1); and telomerase (i.e., an enzyme that adds DNA to the ends of telomeres; telomerase may be used to elongate telomeres and prevent them from shortening).

In some embodiments, the dose of the pan-cancer phenotype therapeutic agents that treat, prevent, or inhibit pan-cancer phenotype can be decreased by about 10%, by about 20%, by about 30%, by about 40%, by about 50%, by about 60%, by about 70%, by about 80%, or by about 90% for subjects that are heterozygous for an RTEL1 variant nucleic acid molecule or RTEL1 reference (i.e., a less than the standard dosage amount) compared to subjects that are homozygous for an RTEL1 variant nucleic acid molecule (who may receive a standard dosage amount). In some embodiments, the dose of the pan-cancer phenotype therapeutic agents that treat, prevent, or inhibit pan-cancer phenotype can be decreased by about 10%, by about 20%, by about 30%, by about 40%, or by about 50%. In some embodiments, the dose of the pan-cancer phenotype therapeutic agents that treat, prevent, or inhibit pan-cancer phenotype can be decreased by about 10%, by about 20%, by about 30%, by about 40%, by about 50%, by about 60%, by about 70%, by about 80%, or by about 90% for subjects that are heterozygous for an RTEL1 variant nucleic acid molecule or RTEL1 reference compared to subjects that are RTEL1 reference. In addition, subjects that are heterozygous for an RTEL1 variant nucleic acid molecule or RTEL1 reference can be administered the pan-cancer phenotype therapeutic agents less frequently compared to subjects that are heterozygous for the RTEL1 variant nucleic acid molecule.

Administration of the pan-cancer phenotype therapeutic agents that treat, prevent, or inhibit pan-cancer phenotype and/or RTEL1 inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the pan-cancer phenotype therapeutic agents and/or RTEL1 inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients, or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in pan-cancer phenotype, a decrease/reduction in the severity of pan-cancer phenotype (such as, for example, a reduction or inhibition of development of pan-cancer phenotype), a decrease/reduction in symptoms and disease-related effects, delaying the onset of symptoms and disease-related effects, reducing the severity of symptoms of disease-related effects, reducing the number of symptoms and disease-related effects, reducing the latency of symptoms and disease-related effects, an amelioration of symptoms and disease-related effects, reducing secondary symptoms, reducing secondary infections, preventing relapse to pan-cancer phenotype, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of pan-cancer phenotype development/progression (such as, for example, a complete or partial avoidance/inhibition or a delay), and an increased survival time of the affected host animal, following administration of a therapeutic protocol. Treatment of pan-cancer phenotype encompasses the treatment of a subject already diagnosed as having any form of pan-cancer phenotype at any clinical stage or manifestation, the delay of the onset or evolution or aggravation or deterioration of the symptoms or signs of pan-cancer phenotype, and/or preventing and/or reducing the severity of pan-cancer phenotype.

In some embodiments, the RTEL1 inhibitor and the pan-cancer phenotype therapeutic agent are disposed within a pharmaceutical composition. In some embodiments, the RTEL1 inhibitor is disposed within a first pharmaceutical composition and the pan-cancer phenotype therapeutic agent is disposed within a second pharmaceutical composition. In some embodiments, the first pharmaceutical composition and the second pharmaceutical composition are administered simultaneously. In some embodiments, the first pharmaceutical composition is administered before the second pharmaceutical composition. In some embodiments, the first pharmaceutical composition is administered after the second pharmaceutical composition.

The present disclosure also provides methods of identifying a subject having an increased risk of developing pan-cancer phenotype. In some embodiments, the method comprises determining or having determined in a biological sample obtained from the subject the presence or absence of an RTEL1 variant nucleic acid molecule (such as a genomic nucleic acid molecule, mRNA molecule, and/or cDNA molecule). When the subject lacks an RTEL1 variant nucleic acid molecule (i.e., the subject is genotypically categorized as RTEL1 reference), then the subject has an increased risk of developing pan-cancer phenotype. When the subject has an RTEL1 variant nucleic acid molecule (i.e., the subject is heterozygous or homozygous for an RTEL1 variant nucleic acid molecule), then the subject has a decreased risk of developing pan-cancer phenotype. In some embodiments, the RTEL1 variant nucleic acid molecule is a RTEL1 variant genomic nucleic acid molecule that comprises the genetic variation listed in Table 1, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63687647:T:G, 20:63687688:C:G, 20:63693211:C:T, 20:63685789:A:C, 20:63662841:C:T, 20:63695551:CTG:C, 20:63667489:C:T, 20:63694876:C:T, 20:63661341:C:T, 20:63688315:G:A, 20:63662559:G:C, 20:63690137:A:C, 20:63667492:G:A, 20:63678268:T:C, 20:63690835:G:T, 20:63694489:G:A, 20:63687706:C:T, and 20:63667554:G:T, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63667554:G:T, 20:63685789:A:C, 20:63693211:C:T (rs398123017), 20:63694489:G:A, and 20:63695551:CTG:C (rs1316931773), or an mRNA molecule produced therefrom, or a cDNA molecule produced from the mRNA molecule.

Having a single copy of an RTEL1 variant nucleic acid molecule is more protective of a subject from developing pan-cancer phenotype than having no copies of an RTEL1 variant nucleic acid molecule. Without intending to be limited to any particular theory or mechanism of action, it is believed that a single copy of an RTEL1 variant nucleic acid molecule (i.e., heterozygous for an RTEL1 variant nucleic acid molecule) is protective of a subject from developing pan-cancer phenotype and it is also believed that having two copies of an RTEL1 variant nucleic acid molecule (i.e., homozygous for an RTEL1 variant nucleic acid molecule) may be more protective of a subject from developing pan-cancer phenotype, relative to a subject with a single copy. Thus, in some embodiments, a single copy of an RTEL1 variant nucleic acid molecule may not be completely protective, but instead, may be partially or incompletely protective of a subject from developing pan-cancer phenotype. While not desiring to be bound by any particular theory, there may be additional factors or molecules involved in the development of pan-cancer phenotype that are still present in a subject having a single copy of an RTEL1 variant nucleic acid molecule, thus resulting in less than complete protection from the development of pan-cancer phenotype.

Determining whether a subject has an RTEL1 variant nucleic acid molecule in a biological sample from a subject and/or determining whether a subject has an RTEL1 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing pan-cancer phenotype, the subject is administered a pan-cancer phenotype therapeutic agent, and/or an RTEL1 inhibitor, as described herein. For example, when the subject is RTEL1 reference, and therefore has an increased risk of developing pan-cancer phenotype, the subject is administered a pan-cancer phenotype therapeutic agent in an amount that is the same as or less than a standard dosage amount, and/or is administered an RTEL1 inhibitor. In some embodiments, when the subject is heterozygous for an RTEL1 variant nucleic acid molecule, the subject is administered the pan-cancer phenotype therapeutic agent in an amount that is the same as or less than a standard dosage amount, and/or is administered an RTEL1 inhibitor. In some embodiments, when the subject is homozygous for an RTEL1 variant nucleic acid molecule, the subject is administered a pan-cancer phenotype therapeutic agent in a standard dosage amount. In some embodiments, the subject is RTEL1 reference. In some embodiments, the subject is heterozygous for an RTEL1 variant nucleic acid molecule. In some embodiments, the subject is homozygous for an RTEL1 variant nucleic acid molecule.

The present disclosure also provides methods of determining a subject's aggregate burden, or risk score, of having two or more RTEL1 variant nucleic acid molecules, and/or two or more RTEL1 variant polypeptides associated with a decreased risk of developing pan-cancer phenotype. The aggregate burden is the sum of two or more genetic variants that can be carried out in an association analysis with pan-cancer phenotype. In some embodiments, the subject is homozygous for one or more RTEL1 variant nucleic acid molecules associated with a decreased risk of developing pan-cancer phenotype. In some embodiments, the subject is heterozygous for one or more RTEL1 variant nucleic acid molecules associated with a decreased risk of developing pan-cancer phenotype. When the subject has a lower aggregate burden, the subject has an increased risk of developing pan-cancer phenotype, and the subject is administered or continued to be administered the pan-cancer phenotype therapeutic agent in an amount that is the same as or less than the standard dosage amount, and/or an RTEL1 inhibitor. When the subject has a higher aggregate burden, the subject has a decreased risk of developing pan-cancer phenotype and the subject is administered or continued to be administered the pan-cancer phenotype therapeutic agent in a standard dosage amount. The higher the aggregate burden, the lower the risk of developing pan-cancer phenotype.

In some embodiments, a subject's aggregate burden of having any two or more RTEL1 variant nucleic acid molecules represents a weighted sum of a plurality of any of the RTEL1 variant nucleic acid molecules. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the RTEL1 gene, where the genetic burden is the number of alleles multiplied by the association estimate with pan-cancer phenotype or related outcome for each allele (e.g., a weighted polygenic burden score). In some embodiments, when the subject has an aggregate burden higher than a desired threshold score, the subject has a decreased risk of developing pan-cancer phenotype. In some embodiments, when the subject has an aggregate burden lower than a desired threshold score, the subject has an increased risk of developing pan-cancer phenotype.

In some embodiments, the aggregate burden may be divided into quintiles, e.g., top quintile, second quintile, intermediate quintile, fourth quintile, and bottom quintile, wherein the top quintile of aggregate burden corresponds to the lowest risk group and the bottom quintile of aggregate burden corresponds to the highest risk group. In some embodiments, a subject having a higher aggregate burden comprises the highest weighted aggregate burdens, including, but not limited to the top 10%, top 20%, top 30%, top 40%, or top 50% of aggregate burdens from a subject population. In some embodiments, the genetic variants comprise the genetic variants having association with pan-cancer phenotype in the top 10%, top 20%, top 30%, top 40%, or top 50% of p-value range for the association. In some embodiments, each of the identified genetic variants comprise the genetic variants having association with pan-cancer phenotype with p-value of no more than about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with pan-cancer phenotype with p-value of less than 5×10⁻⁸. In some embodiments, the identified genetic variants comprise genetic variants having association with pan-cancer phenotype in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects have aggregate burdens in the bottom decile, quintile, or tertile in a reference population. The threshold of the aggregate burden can be determined on the basis of the nature of the intended practical application and the risk difference that would be considered meaningful for that practical application.

In embodiments where the aggregate burden is determined for RTEL1 genetic variants associated with pan-cancer phenotype, then the aggregate burden represents a subject's risk score for developing pan-cancer phenotype. In some embodiments, the aggregate burden or risk score includes the RTEL1 variant genomic nucleic acid molecule that comprises the genetic variation listed in Table 1, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63687647:T: G, 20:63687688:C:G, 20:63693211:C:T, 20:63685789:A:C, 20:63662841:C:T, 20:63695551:CTG:C, 20:63667489:C:T, 20:63694876:C:T, 20:63661341:C:T, 20:63688315:G:A, 20:63662559:G:C, 20:63690137:A:C, 20:63667492:G:A, 20:63678268:T:C, 20:63690835:G:T, 20:63694489:G:A, 20:63687706:C:T, and 20:63667554:G:T, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63667554:G:T, 20:63685789:A:C, 20:63693211:C:T (rs398123017), 20:63694489:G:A, and 20:63695551:CTG:C (rs1316931773), or an mRNA molecule produced therefrom, or a cDNA molecule produced from the mRNA molecule. In some embodiments, a subject's aggregate burden can be determined for RTEL1 genetic variants associated with pan-cancer phenotype in combination with additional genetic variants for other genes also associated with pan-cancer phenotype to produce a polygenic risk score (PRS) for developing pan-cancer phenotype. In some embodiments, the PRS includes the RTEL1 variant genomic nucleic acid molecule that comprises the genetic variation listed in Table 1, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63687647:T:G, 20:63687688:C:G, 20:63693211:C:T, 20:63685789:A:C, 20:63662841:C:T, 20:63695551:CTG:C, 20:63667489:C:T, 20:63694876:C:T, 20:63661341:C:T, 20:63688315:G:A, 20:63662559:G:C, 20:63690137:A:C, 20:63667492:G:A, 20:63678268:T:C, 20:63690835:G:T, 20:63694489:G:A, 20:63687706:C:T, and 20:63667554:G:T, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63667554:G:T, 20:63685789:A:C, 20:63693211:C:T (rs398123017), 20:63694489:G:A, and 20:63695551:CTG:C (rs1316931773), or an mRNA molecule produced therefrom, or a cDNA molecule produced from the mRNA molecule.

The present disclosure also provides methods of detecting the presence or absence of an RTEL1 variant nucleic acid molecule (i.e., a genomic nucleic acid molecule, an mRNA molecule, or a cDNA molecule produced from an mRNA molecule) in a biological sample from a subject. It is understood that gene sequences within a population and mRNA molecules encoded by such genes can vary due to polymorphisms such as single-nucleotide polymorphisms.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any RTEL1 variant nucleic acid molecule, preliminary processing designed to isolate or enrich the biological sample for the genomic DNA can be employed. A variety of techniques may be used for this purpose. When detecting the level of any RTEL1 variant nucleic acid molecule, different techniques can be used enrich the biological sample with mRNA molecules. Various methods to detect the presence or level of an mRNA molecule or the presence of a particular variant genomic DNA locus can be used.

In some embodiments, detecting an RTEL1 variant nucleic acid molecule in a subject comprises performing a sequence analysis on a biological sample obtained from the subject to determine whether an RTEL1 genomic nucleic acid molecule in the biological sample, and/or an RTEL1 mRNA molecule in the biological sample, and/or an RTEL1 cDNA molecule produced from an mRNA molecule in the biological sample, is present in the sample. In some embodiments, the methods detect the RTEL1 variant genomic nucleic acid molecule that comprises the genetic variation listed in Table 1, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63687647:T:G, 20:63687688:C:G, 20:63693211:C:T, 20:63685789:A:C, 20:63662841:C:T, 20:63695551:CTG:C, 20:63667489:C:T, 20:63694876:C:T, 20:63661341:C:T, 20:63688315:G:A, 20:63662559:G:C, 20:63690137:A:C, 20:63667492:G:A, 20:63678268:T:C, 20:63690835:G:T, 20:63694489:G:A, 20:63687706:C:T, and 20:63667554:G:T, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63667554:G:T, 20:63685789:A:C, 20:63693211:C:T (rs398123017), 20:63694489:G:A, and 20:63695551:CTG:C (rs1316931773), or an mRNA molecule produced therefrom, or a cDNA molecule produced from the mRNA molecule.

In some embodiments, the methods of detecting the presence or absence of an RTEL1 variant nucleic acid molecule (such as, for example, a genomic nucleic acid molecule, an mRNA molecule, and/or a cDNA molecule produced from an mRNA molecule) in a subject comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising an RTEL1 genomic nucleic acid molecule or mRNA molecule, and if mRNA, optionally reverse transcribing the mRNA into cDNA. Such assays can comprise, for example determining the identity of these positions of the particular RTEL1 nucleic acid molecule. In some embodiments, the method is an in vitro method.

In some embodiments, the determining step, detecting step, or sequence analysis comprises sequencing at least a portion of the nucleotide sequence of the RTEL1 genomic nucleic acid molecule, the RTEL1 mRNA molecule, or the RTEL1 cDNA molecule in the biological sample that comprises a genetic variation compared to the corresponding RTEL1 reference molecule. In some embodiments, the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only an RTEL1 genomic nucleic acid molecule is analyzed. In some embodiments, only an RTEL1 mRNA is analyzed. In some embodiments, only an RTEL1 cDNA obtained from the RTEL1 mRNA is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to an RTEL1 variant genomic sequence, variant mRNA sequence, or variant cDNA sequence and not the corresponding RTEL1 reference sequence under stringent conditions and determining whether hybridization has occurred.

In some embodiments, the determining step, detecting step, or sequence analysis comprises: a) amplifying at least a portion of the RTEL1 nucleic acid molecule that encodes the RTEL1 polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an RTEL1 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to RTEL1 variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to RTEL1 variant nucleic acid molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the RTEL1 variant nucleic acid molecules disclosed herein. The primers described herein can be used to amplify any RTEL1 variant nucleic acid molecule, or a fragment thereof.

In the context of the disclosure “specifically hybridizes” means that the probe or primer (such as, for example, the alteration-specific probe or alteration-specific primer) does not hybridize to a nucleic acid sequence encoding an RTEL1 reference genomic nucleic acid molecule, an RTEL1 reference mRNA molecule, and/or an RTEL1 reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×his or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides pan-cancer phenotype therapeutic agents that treat, prevent, or inhibit pan-cancer phenotype for use in the treatment or prevention of pan-cancer phenotype in a subject having an RTEL1 variant nucleic acid molecule. Any of the pan-cancer phenotype therapeutic agents that treat, prevent, or inhibit pan-cancer phenotype described herein can be used herein. Any of the RTEL1 variant nucleic acid molecules disclosed herein can be used herein. In some embodiments, the RTEL1 variant nucleic acid molecule is a RTEL1 variant genomic nucleic acid molecule that comprises the genetic variation listed in Table 1, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63687647:T:G, 20:63687688:C:G, 20:63693211:C:T, 20:63685789:A:C, 20:63662841:C:T, 20:63695551:CTG:C, 20:63667489:C:T, 20:63694876:C:T, 20:63661341:C:T, 20:63688315:G:A, 20:63662559:G:C, 20:63690137:A:C, 20:63667492:G:A, 20:63678268:T:C, 20:63690835:G:T, 20:63694489:G:A, 20:63687706:C:T, and 20:63667554:G:T, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63667554:G:T, 20:63685789:A:C, 20:63693211:C:T (rs398123017), 20:63694489:G:A, and 20:63695551:CTG:C (rs1316931773), or an mRNA molecule produced therefrom, or a cDNA molecule produced from the mRNA molecule.

The present disclosure also provides pan-cancer phenotype therapeutic agents that treat, prevent, or inhibit pan-cancer phenotype for use in the preparation of a medicament for treating or preventing pan-cancer phenotype in a subject having an RTEL1 variant nucleic acid molecule. Any of the pan-cancer phenotype therapeutic agents that treat, prevent, or inhibit pan-cancer phenotype described herein can be used herein. Any of the RTEL1 variant nucleic acid molecules disclosed herein can be used herein. In some embodiments, the RTEL1 variant nucleic acid molecule is a RTEL1 variant genomic nucleic acid molecule that comprises the genetic variation listed in Table 1, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63687647:T:G, 20:63687688:C:G, 20:63693211:C:T, 20:63685789:A:C, 20:63662841:C:T, 20:63695551:CTG:C, 20:63667489:C:T, 20:63694876:C:T, 20:63661341:C:T, 20:63688315:G:A, 20:63662559:G:C, 20:63690137:A:C, 20:63667492:G:A, 20:63678268:T:C, 20:63690835:G:T, 20:63694489:G:A, 20:63687706:C:T, and 20:63667554:G:T, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63667554:G:T, 20:63685789:A:C, 20:63693211:C:T (rs398123017), 20:63694489:G:A, and 20:63695551:CTG:C (rs1316931773), or an mRNA molecule produced therefrom, or a cDNA molecule produced from the mRNA molecule.

The present disclosure also provides RTEL1 inhibitors for use in the treatment or prevention of pan-cancer phenotype in a subject that is RTEL1 reference or is heterozygous for an RTEL1 variant nucleic acid molecule. Any of the RTEL1 inhibitors described herein can be used herein. Any of the RTEL1 variant nucleic acid molecules disclosed herein can be used herein. In some embodiments, the RTEL1 variant nucleic acid molecule is a RTEL1 variant genomic nucleic acid molecule that comprises the genetic variation listed in Table 1, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63687647:T:G, 20:63687688:C:G, 20:63693211:C:T, 20:63685789:A:C, 20:63662841:C:T, 20:63695551:CTG:C, 20:63667489:C:T, 20:63694876:C:T, 20:63661341:C:T, 20:63688315:G:A, 20:63662559:G:C, 20:63690137:A:C, 20:63667492:G:A, 20:63678268:T:C, 20:63690835:G:T, 20:63694489:G:A, 20:63687706:C:T, and 20:63667554:G:T, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63667554:G:T, 20:63685789:A:C, 20:63693211:C:T (rs398123017), 20:63694489:G:A, and 20:63695551:CTG:C (rs1316931773), or an mRNA molecule produced therefrom, or a cDNA molecule produced from the mRNA molecule.

The present disclosure also provides RTEL1 inhibitors in the preparation of a medicament for treating or preventing pan-cancer phenotype in a subject that is RTEL1 reference or is heterozygous for an RTEL1 variant nucleic acid molecule. Any of the RTEL1 inhibitors described herein can be used herein. Any of the RTEL1 variant nucleic acid molecules disclosed herein can be used herein. In some embodiments, the RTEL1 variant nucleic acid molecule is a RTEL1 variant genomic nucleic acid molecule that comprises the genetic variation listed in Table 1, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63687647:T:G, 20:63687688:C:G, 20:63693211:C:T, 20:63685789:A:C, 20:63662841:C:T, 20:63695551:CTG:C, 20:63667489:C:T, 20:63694876:C:T, 20:63661341:C:T, 20:63688315:G:A, 20:63662559:G:C, 20:63690137:A:C, 20:63667492:G:A, 20:63678268:T:C, 20:63690835:G:T, 20:63694489:G:A, 20:63687706:C:T, and 20:63667554:G:T, or an mRNA molecule produced therefrom, or a cDNA molecule produced from the mRNA molecule. In some embodiments, the RTEL1 variant nucleic acid molecule may comprise any one or more of the following genetic variations in the genomic nucleic acid molecule (referring to the chromosome: positions set forth in the GRCh38/hg38 human genome assembly): 20:63667554:G:T, 20:63685789:A:C, 20:63693211:C:T (rs398123017), 20:63694489:G:A, and 20:63695551:CTG:C (rs1316931773), or an mRNA molecule produced therefrom, or a cDNA molecule produced from the mRNA molecule.

In some embodiments, the RTEL1 inhibitor and the pan-cancer phenotype therapeutic agent are disposed within a pharmaceutical composition. In some embodiments, the RTEL1 inhibitor is disposed within a first pharmaceutical composition and the pan-cancer phenotype therapeutic agent is disposed within a second pharmaceutical composition. In some embodiments, the first pharmaceutical composition and the second pharmaceutical composition are administered simultaneously. In some embodiments, the first pharmaceutical composition is administered before the second pharmaceutical composition. In some embodiments, the first pharmaceutical composition is administered after the second pharmaceutical composition.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: General Methods

### Study Cohorts and Samples

High quality whole-exome sequencing data as well as phenotypes were generated from linked electric health records (EHRs) for 947,128 individuals across eight biobanks. These biobanks consisted of the UK Biobank (Bycroft et al., Nature, 2018, 562, 203-209), the Geisinger Health System (Carey et al., Genetics in Medicine, 2016, 18, 906-913), the Penn Medicine Biobank (Verma et al., J. Personal. Med., 2022, 12, 1974), the BioMe BioBank from Mount Sinai (Gottesman et al., Clin. Pharmacol. Therap., 2013, 94, 214-217), the Malmo Population-Based cohort (Berglund et al., J. Int. Med., 1993, 233, 45-51), The Mayo Clinic Biobank (Olson et al., Mayo Clinic Proc., 2013, 88, 952-962; and Olson et al., Bmj Open 9, 2019, e032707), the UCLA ATLAS Community Health Initiative (Johnson et al., Cell Genom., 2023, 3), and The Biobank at the Colorado Center for Personalized Medicine (Wiley et al, Building a Vertically-Integrated Genomic Learning Health System: The Colorado Center for Personalized Medicine Biobank, 2022, medRxiv 2022.06. 09.22276222). These cohorts generally represent a reasonable cross-section of the general population. Further, these biobank cohorts represent individuals from multiple countries (e.g., Sweden, United Kingdom, United States), and multiple ancestral backgrounds (e.g., European, African).

### Exome Sequencing and Variant Calling

Sample preparation and sequencing were performed as previously described (Backman et al., Nature, 2021, 1-10; and Van Hout et al., Nature, 2020, 586, 749-756). Briefly, sequencing libraries were prepped using genomic DNA samples collected from each of eight population-based initiatives, followed by multiplexed exome capture and sequencing. Sequencing was performed on the Illumina NovaSeq 6000 platform using S2 or S4 flow cells. Read mapping, variant calling, and quality control were carried out according to the SPB protocol described (Van Hout et al., Nature, 2020, 586, 749-756), which included the mapping of reads to the hg38 reference genome with BWA MEM, the identification of small variants with WeCall, and the use of GLnexus to aggregate these files into joint-genotyped, multi-sample VCF files. While certain UKB exome analysis efforts have used calls generated with the OQFE pipeline (Szustakowski et al., Nat. Genet., 2021, 53, 942-948), this pipeline has only been used to a limited degree for disease association analysis. Therefore, calls from the SBP pipeline were used, which have been used very extensively for disease association analysis, including the largest sets of association analyses carried out with UKB exome data (Kessler et al., Nature, 2022, 612, 301-309; and Akbari et al., Science, 2021, 373, eabf8683). Depth and allelic valance filters were then applied, and samples were filtered out if they showed disagreement between genetically-determined and reported sex, high rates of heterozygosity/contamination (VBID>5%), low sequence coverage, or genetically-determined sample duplication.

### Defining Cancer Phenotypes

To define the cancer phenotypes (pan-cancer and cancer type specific), a combination of cancer registry data, hospital inpatient (HESIN) data, and data from general practitioner (GP) records was used to ascertain ICD10 codes. The majority of the cancer data came from the cancer registry, which was supplemented with the other sources. The cancer type specific phenotypes were defined using ICD-10 Codes. Given differences in disease etiology between non-melanoma skin cancer (NMSC) and other (solid) cancers, and the significant impact of sun-exposure and skin color on NMSC risk, the pan-cancer phenotype used for the main analyses did not consider individuals as cases on the basis of a diagnoses of NMSC. A more inclusive phenotype (all_malignant) that did include individuals with diagnoses of NMSC was also defined, and this phenotype was used for sensitivity analyses (result were not meaningfully different). A simplifying assumption that individuals that are missing the EHR-based data necessary for ICD-10 cancer phenotype generation are unlikely to have the relevant cancer was made, and these individuals were used as controls. Overall, after excluding individuals with missing covariate data (e.g., age, sex, etc) from the regression-based genetic testing, a maximum of 746,223 individuals (e.g., the most inclusive pan-cancer phenotype) were used for genetic association analysis. A number were built from multiple codes or were stratified by sex, including the pan-cancer phenotypes, a malignant blood phenotype, a phenotype that included both bone and cartilage cancers, and a phenotype that aggregated oropharyngeal cancers. Codes reflecting early stage and/or benign cancerous disease for adrenal, cervical, and thyroid phenotypes were also used given that these cancers are often biopsied/resected as part of surveillance and diagnosis.

### Genetic Association Analyses

To perform genetic association analyses, the genome-wide regression approach implemented in REGENIE (Mbatchou et al., Computationally efficient whole-genome regression for quantitative and binary traits, 2021) was used, as described (Backman et al., Nature, 2021, 1-10). Briefly, regressions were run separately for data derived from exome-sequencing as well as data derived from genetic imputation using TOPMed (Gu et al., J. Cancer, 2019, 10, 6170), and results were combined across these data sources for downstream analysis. Step 1 of REGENIE uses genetic data to predict individual values for the trait of interest (i.e., a PRS), which is then used as a covariate in step 2 to adjust for population structure and other potential confounding. For step 1, variants from array data with a minor allele frequency (MAF)>1%, <10% missingness, Hardy-Weinberg equilibrium test P-value>10⁻¹⁵ and linkage disequilibrium (LD) pruning (1000 variant windows, 100 variant sliding windows and r²<0.9) were used, and any variants with high inter-chromosomal LD, in the major histocompatibility (MHC) region, or in regions of low complexity were excluded. For association analyses in step 2 of REGENIE, age, age², sex, and age-by-sex, and 10 ancestry-informative principal components (PCs) were used as covariates. For analyses involving exome data, an indicator variable representing exome sequencing batch, and 20 PCs derived from the analysis of rare exomic variants (MAF between 2.6×10⁻⁵ and 0.01) were also included as covariates. Genome-wide significance cutoffs and rare variant burden testing were calibrated according to the logic outlined and results reported by Backman et al. (Nature, 2021, 599, 628-634). Briefly, P≤5×10⁻⁸ and P≤7.14×10⁻¹⁰, were used as the cutoffs for common and rare variant associations, respectively. While P≤3.6×10⁻⁷ was used by Backman et al. as the cutoff for genome-wide significant gene burden associations, this was viewed as too strict for the gene burden testing performed here, which was carried out to evaluate rare variants in a gene (i.e., RTEL1) already implicated by common variant signal. Therefore, based on the enrichment analyses presented by Backman et al., P≤1×10⁻⁴ was used as a significance cutoff. Results were visualized and processed using an in-house version of the FUMA software (Watanabe et al., Nature Commun., 2017, 8, 1-11). Association analyses were performed separately for different continental ancestries defined based on the array data, as described (Backman et al., Nature, 2021, 1-10).

### Phenome-Wide Association Study (PheWAS)

GWAS and ExWAS were performed for 2,271 binary traits defined across cohorts, and then meta analyzed these single cohort-derived statistical results across cohorts to generate a final set of genetic association results. These association results were queried for RTEL1 common variants prioritized by finemapping (i.e., all variants in the 95% credible set for each of 9 independent signals), as well as gene burden association results from aggregating putative loss-of-function variants (pLoF) variants in RTEL1 with an alternative allele frequency (AAF)<0.001 across the data. This process was repetade for 3031 quantitative traits available from the UK Biobank. Nearly all of the traits analyzed are the same as those for which we previously reported common variant associations (Backman et al., Nature, 2021, 1-10). To briefly redescribe how these phenotypes were generated, ICD10-based phenotypic cases were required to have one or more records of diagnosis in the electronic health records, death registry data implicating the disease, or two or more diagnosis in outpatient data mapped to ICD10. For non-ICD10 phenotypes (quantitative measures, clinical outcomes, survey and touchscreen responses, and imaging derived phenotypes), data was derived from the UKB Showcase for the UK Biobank, and from other analogous data for the other cohorts. Participants who did not meet the case definition for a given ICD10-based phenotype were removed from the analyses if they had one diagnosis code in the outpatient data, and included as controls if they had no diagnosis in the outpatient data.

### Risk Modeling Among RTEL1 Carriers

To perform longitudinal modeling, whole exome sequencing and electronic health record data from 358,954 individuals in the UKB Biobank for whom event time information was available (i.e., date at diagnosis) was used. This information was used to evaluate how time to cancer diagnosis from enrollment time (i.e, time-to-event) differed between RTEL1 ploF carriers and non-carriers. This is likely to be more robust to ascertainment biases (e.g., healthy enrolle bias), and can help to support the robustness of our rare variant associations. Time-to-event was calculated as years from study enrollment date to first cancer diagnosis date for individuals with any solid tumor, and as years from enrollment to last encounter or death date for individuals without any diagnosis of cancer (i.e., this was set as the censoring time). Each individual's first cancer date was defined using ICD-10 code dates (i.e., C00-C75), excluding legacy codes (C27, C28, C29, C42, and C59). Non-melanoma skin cancer (NMSC; C44) was also excluded given how common this cancer is and how confounded its prevalence is by environmental exposure (e.g., sun exposure) and ancestral background (e.g., skin color). Individuals with any cancer diagnosis code recorded prior to study enrollment, without any diagnostic codes at all post enrollment, or without genetic data were removed from the analysis to maintain data homogeneity. The data was also right censored at a follow-up time of 13 years, as 95% of individuals had an event or were censored by that point of follow-up. The final analysis included 42,259 individuals with a cancer a cancer diagnosis within 13 years of study enrollment and 317,695 censored patients.

The effects of RTEL1 pLoF variants on time to first cancer diagnosis was tested using Cox proportional hazard modeling. Models included 263 RTEL1 pLoF carriers (20 of whom had cancer event among 41,239 that developed a cancer over the event time (7.6%)). Age at enrollment, sex, smoking history, and the top ten genetically determined principal components were included as covariates in the models.

### Example 2: Association of Pan-Cancer Phenotype and RTEL1 pLoFs

### Genetic Associations Between RTEL1 Variants and a Pan-Cancer Phenotype

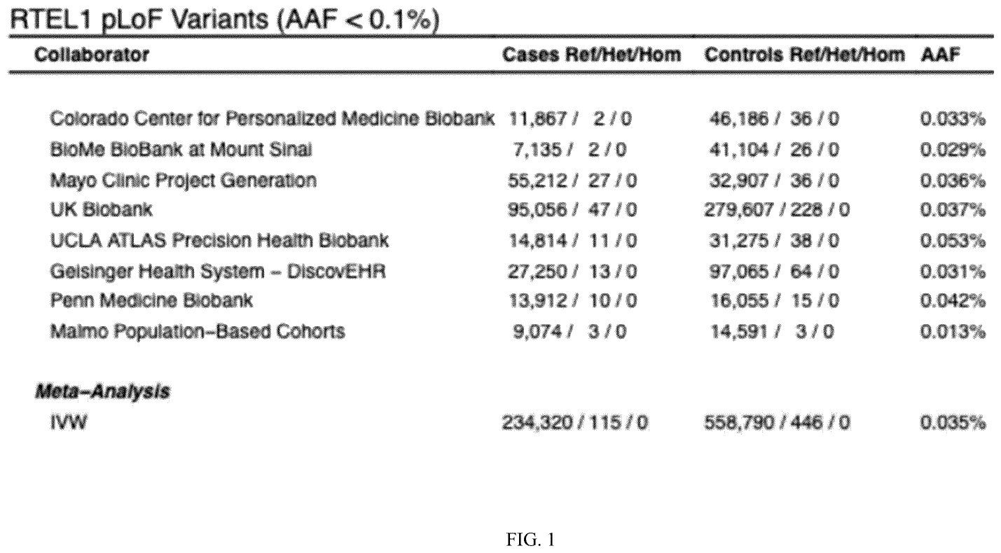

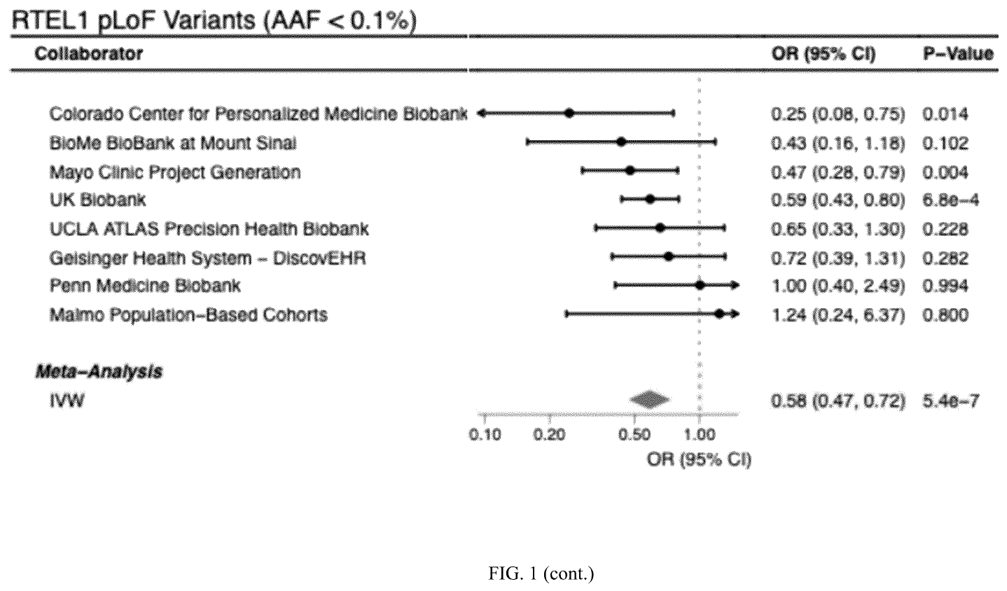

A pan-cancer phenotype was generated in 947,128 individuals across eight biobanks and common and rare variant genetic associations analysis was performed using whole genome regression (Mbatchou et al., Computationally efficient whole-genome regression for quantitative and binary traits, 2021). A strong common variant driven association “peak”, with the index variant (i.e., lead signal, 20:63695226:G:T, OR=1.06 (1.05-1.07), P=3.4×10⁻¹⁷) falling within a splice region of the RTEL1 gene, was identified. This common variant driven association peak was then finemapped and 9 independent signals were identified. These signals reflect the genetic complexity at this locus, with credible set leading variants including those that are intronic, downstream, upstream, in splice regions, and protein altering relative to RTEL1. These independent signals go in opposing directions (4 risk increasing and 5 risk reducing), which further underscores the difficulty of determining from these common variant associations any potential direction of effect at the gene level. To address this, association between rare functional variants (AAF <0.001) and pan-cancer phenotype was examined in a gene burden framework (Backman et al., Nature, 2021, 1-10). Notably, when meta-analyzing results across cohorts, a protective association (OR=0.54, P=1.90×10⁻⁶) was identified with a gene burden of predicted loss of function (pLoF) variants (FIG. 1). This analysis included 437 pLoF variants (73 in cases), and the identified signal was consistent across cohorts, with nominal significance reached (P<=0.1) in three (UCLA, MAYO, UKB) of the eight cohorts. This gene burden association signal suggests that the common variant association signals at this locus are in fact driven by RTEL1, and that loss of RTEL1 function is significantly protective against cancer.

Genetic Associations Between RTEL1 pLoF Variants and Individual Cancer Types

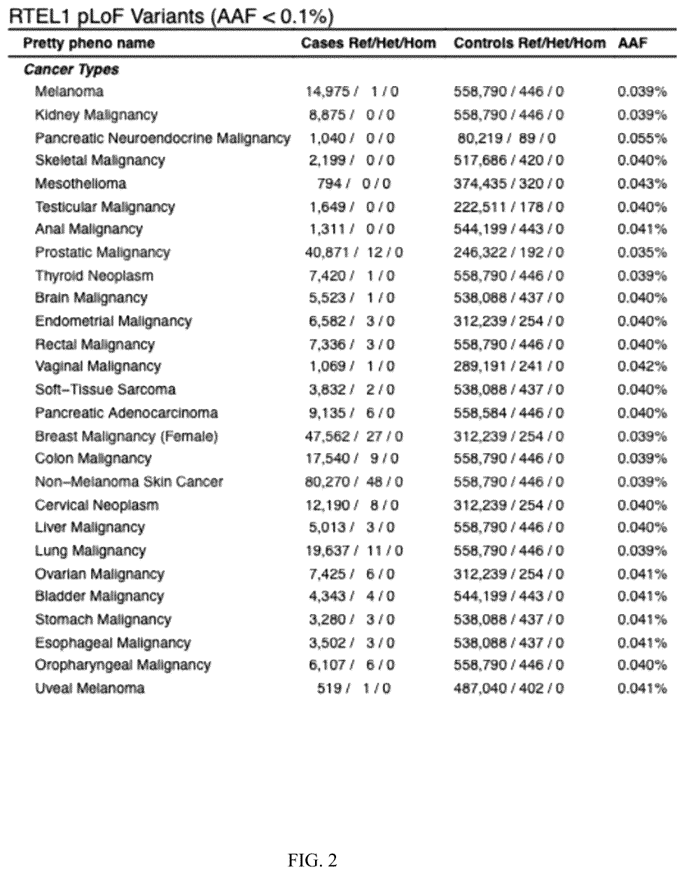

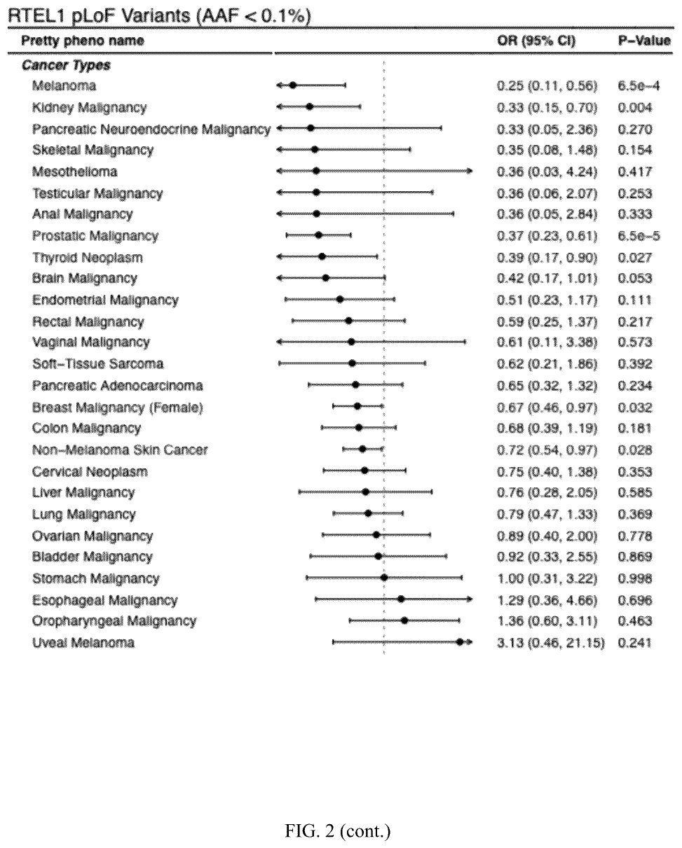

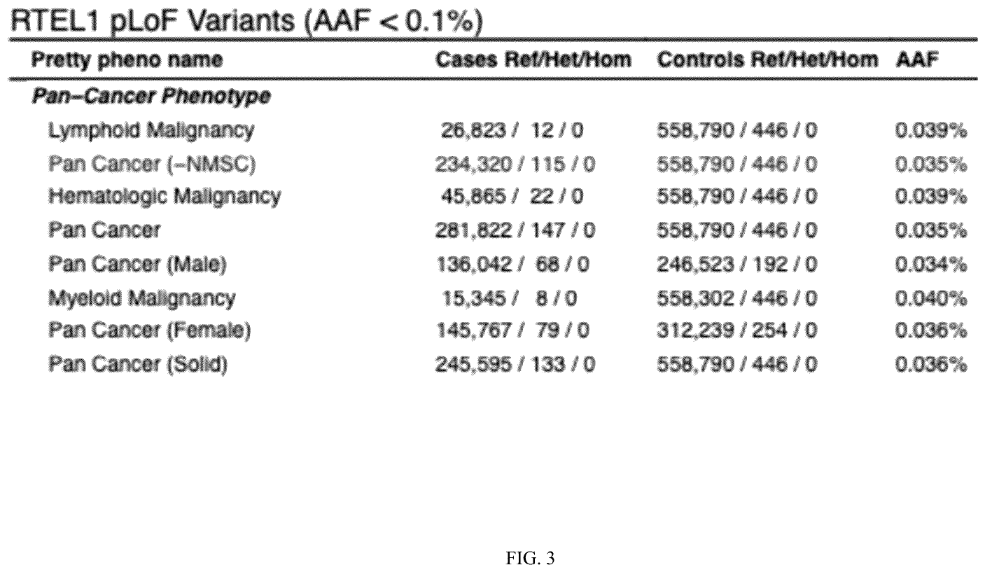

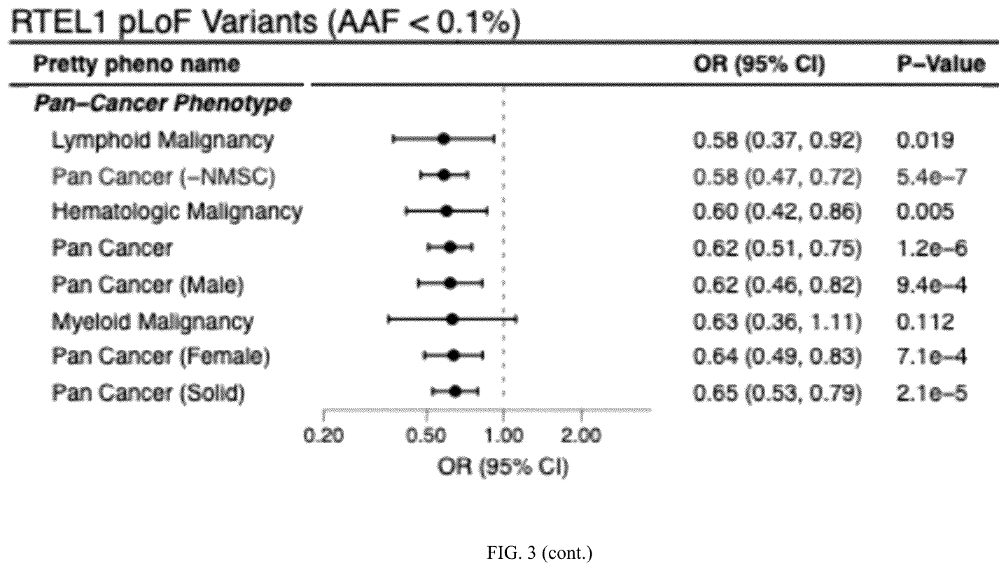

To further dissect this protective pan-cancer association signal, the association between the RTEL1 pLoF gene burden and 23 cancer type-specific phenotypes was examined (i.e., all malignant, all malignant except NMSC, all malignant female, all malignant male, adrenal, bladder, bone/cartilage, brain, breast, cervical, colon, esophagus, kidney, lip/oral cavity/pharynx, liver, lung, melanoma, non-melanoma skin cancer, ovarian, pancreatic, prostate, rectal, stomach, testicular, and thyroid; FIG. 2). 17/23 of these associations were in a protective direction (OR<=0.95), with seven reaching nominal significance (P<0.1), while only oropharyngeal cancer showed nominally significant association with increased risk. Interestingly, among cancer-type-specific analyses, RTEL1 pLoF variant carrier status was most significantly associated with protection from prostate cancer (OR=0.36 (0.20-0.64), P=5.5×10⁻⁴). This raised questions as to whether the pan-cancer association was being largely driven by prostate cancer and/or whether it was male specific. To address this, association between RTEL1 pLoF variants and pan-cancer phenotypes split by sex was examined. RTEL1 pLoF carrier status was significantly associated with a reduced risk of cancer in both females (FIG. 3, Panel A) and males (FIG. 3, Panel B), suggesting that the effect of RTEL1 disruption on cancer risk is not sex specific.

Identifying Individual Rare Variant that Drive Gene Burden Associations Between Cancer Phenotypes and RTEL1

To identify individual rare variants contributing to the protective RTEL1 gene burden signal, an iterative leave one variant out (iLOVO) strategy was employed to identify variants that led to a notable change in p-value when removed from a gene burden coding. Briefly, this analysis was not restricted to pLoF variants, and instead considered pLoF and missense variants. Gene burden codings with varying definitions were tested marginally and the burden type with the most significant p-value was chosen to start the iterative process. Each variant was then removed from the burden definition and the burden was tested for association with the pan-cancer phenotype. In this way, variants that dropped the p-value by a sufficient amount or below a pre-specified p-value threshold were catalogued. After this, orthogonal gene burden definitions comprising other variant types across rarer allele frequency bins were evaluated using the same iterative process. This iLOVO strategy is most effective at identifying variants in larger cohorts, and by employing it, 18 rare variants were identified across the two cohorts with the greatest number of cancer cases (UKB and MAYO). These variants were spread across the RTEL1 protein, suggesting that loss of a variety of RTEL1 domains, including the C-terminal domain, may be sufficient to drive the pan-cancer protective signal observed.

RTEL1 pLoF Carriers have Shorter Telomeres

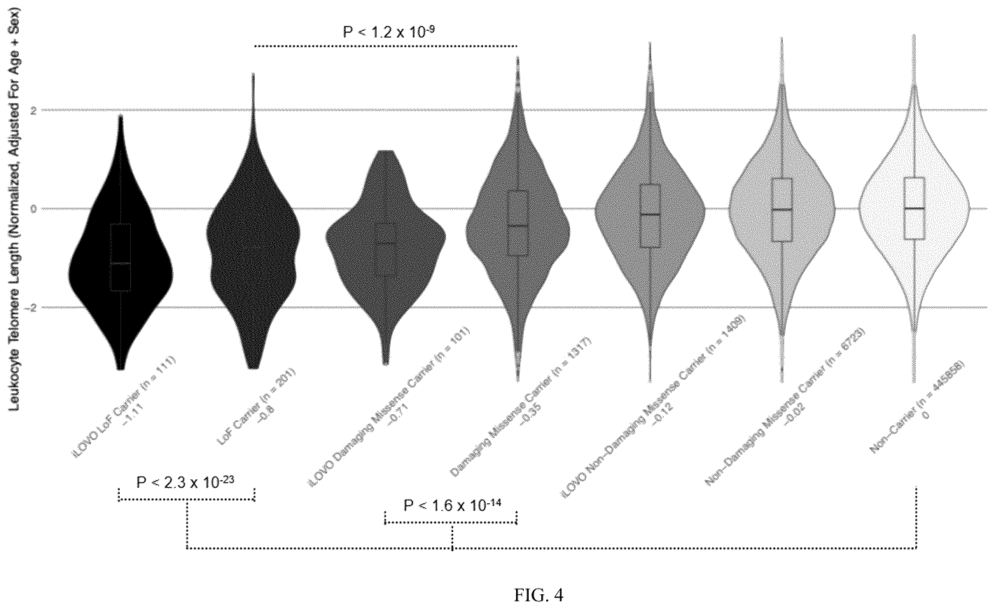

While a recent exome-wide association study of leukocyte telomere length that used the same UKB data employed herein reported that loss-of-function variants in RTEL1 are associated with reduced telomere length (Kessler et al., Nature, 2022, 612, 301-309), it was desired to confirm that relationship using the pLoF definitions and variants employ for the present genetic analyses (as well as for the variants identified herein using the iLOVO procedure). Indeed, after adjusting for age, carriers of RTEL1 pLoF variants had significantly shorter telomeres than non-carriers (P<1.2×10⁻¹², Wilcoxson Rank Sum Test, FIG. 4). In fact, compared with non-carriers of functional RTEL1 variants, telomere length was progressively shorter for those with RTEL1 missense variants predicted to be deleterious by only one computational predictor (i.e., “missense_1”), RTEL1 missense variants predicted to be deleterious by five computational predictors (i.e., “missense_5”), and pLoF variants, respectively. When considering the estimated age effect on telomere length (B=−0.023 normalized units per year of age, model adjusted for sex), the median differences between these groups was equivalent to three years of aging for RTEL1 missense_1 carriers, 9.5 years of aging for RTEL1 missense_5 carriers, and 37 years of aging for pLoF carriers. Carriers of the iLOVO variants identified herein showed reduced telomere lengths consistent with their annotation category.

Time-to-Cancer-Event Modeling Among RTEL1 Rare pLoF Variant Carriers

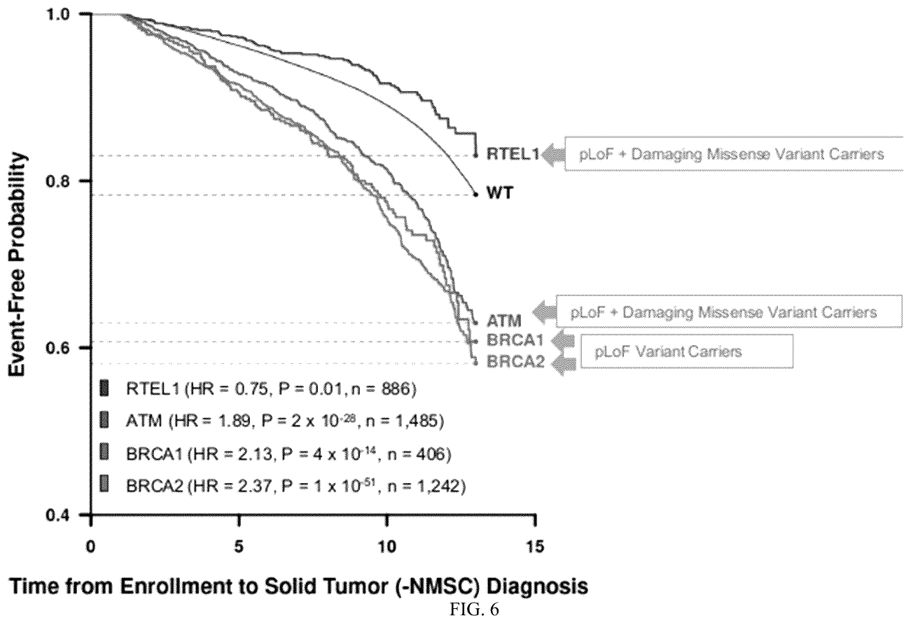

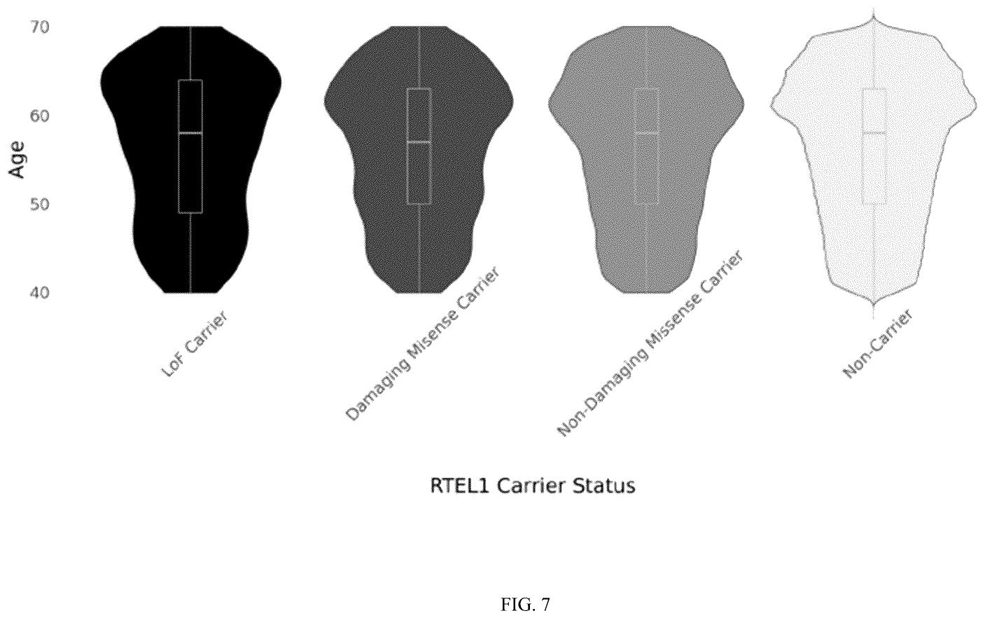

To evaluate whether early death from telomere related disease was confounding the RTEL1 rare variant association signal, time-to-event analysis was performed using data from the UKB. Specifically, the impact of RTEL1 pLoF variants on time from enrollment to first solid cancer (not including non-melanoma skin cancer) was evaluated, and carrier status for protein altering variants in three known cancer risk genes (ATM, BRCA1, BRCA2) was included as positive controls. Age distributions were not different between RTEL1 carriers and non-carriers (FIG. 7, Panel A). In models adjusting for age, sex, smoking history, and genetic relatedness, RTEL1 pLoF carrier status was associated with a nominally significant reduction in time to first cancer (HR=0.69, P=0.10, FIG. 6), which is consistent with the rare variant association signal. As expected, protein altering variants in ATM, BRCA1, and BRCA2 were associated with an increased risk of cancer (P<1×10⁻¹¹). In an association study in various cohorts for congenital disease diagnosis and RTEL1 status (FIG. 7, Panel B), RTEL1 pLoF carrier status was not found to be associated with congenital disease overall (OR=1.00, P=0.81). Only 3/551 (0.54%) carriers of RTEL1 variants across the seven biobanks with congenital data had documented evidence of a diagnosis of dyskeratosis congenita, while 33 carriers (6%) had a diagnosis of any congenital disease. This suggests that heterozygous loss of function of RTEL1 is both viable and consistent with relative healthiness in early age.

### Association Between Genetically Predicted Telomere Length and Cancer Phenotypes

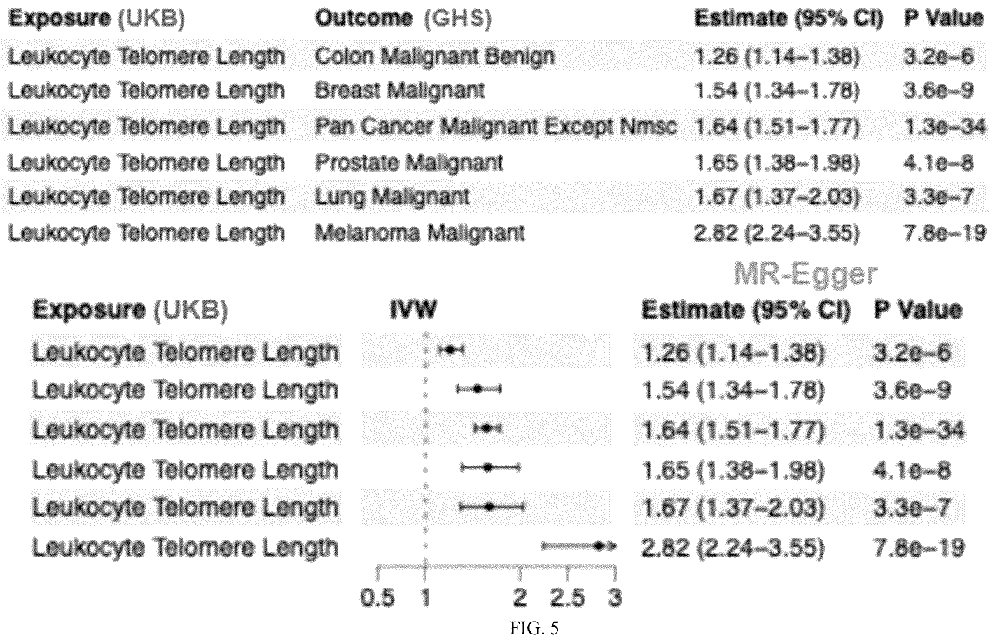

While many studies have used genetic instruments to support a positive relationship between telomere length and cancer risk, it was desired to evaluate the relationship between telomere length and cancer risk using the cancer phenotypes and both common and rare variants as genetic instruments. First, using whole genome regression (Mbatchou et al., Computationally efficient whole-genome regression for quantitative and binary traits, 2021), a GWAS of leukocyte telomere length we performed and independent variants were selected as genetic instrument using conditional iterative analysis. Using these variants, Two-sample Mendelian randomization (MR) was performed, with estimates of instrument effect on cancer coming from analyses performed in the GHS cohort. Specifically, a relationship between telomere length and cancer risk was examined by leveraging results from the analyses of the pan-cancer phenotype as well as the phenotypes for the five most prevalent cancers (breast, prostate, lung, colon, and skin, with skin broken up into non-melanoma and melanoma skin cancers). When considering inverse variance weighted and MR-Egger methods, all results were significance (P<0.05), and provided support for a robust positive relationship between telomere length and cancer risk (FIG. 5). The strongest positive associations were found for melanoma and prostate cancers (i.e., among these common cancers, RTEL1 pLoF carriers showed the largest risk reduction for melanoma and prostate cancer). To evaluate the relation between telomere length and cancer using rare variants, the estimated effects on telomere length and pan-cancer risk was compared for gene burdens of rare pLoF variants (AAF <=0.001) across 38 genes previously reported to directly influence telomere length (Mirabello et al., Human mutation 31, 2010, 1050-1058). Consistent with the MR results when using common variants as genetic instruments, betas estimated for gene burdens by whole genome regression (i.e., ExWAS) were significantly positively correlated (r²=0.519, P=3.5×10⁻⁷). Interestingly, the two genes with the largest negative effects on telomere length and cancer risk were TERT and RTEL, with the former already serving as a target for cancer therapeutic development.

### PheWAS of RTEL1 Variants

To evaluate whether rare RTEL1 pLoF variants influence additional traits beyond telomere length and cancer, a phenome-wide association study (PheWAS) across 5,302 traits derived from the biobank scale clinical data was performed. Binary traits (BTs) were analyzed after defining them across the biobanks and performing meta-analysis, while quantitative traits were tested using data available from the UKB. Trait associations reaching significance after multiple testing correction (P<1×10⁻⁵) included those indicative of telomeropathies, such as blood count changes, pulmonary function results, and diagnosis codes reflective of bone marrow pulmonary disorders. Interestingly, a set of nominally significant associations was discovered between RTEL1 pLoF variants and increased risk of cervical disease (inflammatory/dysplastic diagnostic codes, P<3×10⁻⁴). Perhaps most notably, while a nominal association with congenital cardiac valve insufficiency (OR=6.8 (2.2-21), P=9.5×10⁻⁴) was identified, otherwise, RTEL1 pLoF carrier status was not found to be associated with congenital disease overall (OR=1.00, P=0.81). Only 3/551 (0.54%) carriers of RTEL1 variants across our seven biobanks with congenital data had documented evidence of a diagnosis of dyskeratosis congenita, while 33 carriers (6%) had a diagnosis of any congenital disease. This suggests that heterozygous loss of function of RTEL1 is both viable and consistent with relative healthiness in early age.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having a pan-cancer phenotype or at risk of developing a pan-cancer phenotype, the method comprising administering a Regulator of Telomere Elongation Helicase 1 (RTEL1) inhibitor to the subject, wherein the pan-cancer phenotype comprises adrenal cancer, bladder cancer, bone cancer, cartilage cancer, brain cancer, breast cancer, cervical cancer, colon cancer, esophageal cancer, kidney cancer, oral cavity cancer, pharynx cancer, liver cancer, lung cancer, melanoma, ovarian cancer, pancreatic cancer, prostate cancer, rectal cancer, stomach cancer, testicular cancer, thyroid cancer, or blood cancer.

2-4. (canceled)

5. The method of claim 1, wherein the 1 RTEL1 inhibitor comprises an antisense nucleic acid molecule, a small interfering RNA (siRNA), and/or a short hairpin RNA (shRNA).

6. The method of claim 5, wherein the RTEL1 inhibitor comprises an siRNA.

7. The method of claim 5, wherein the RTEL1 inhibitor comprises an antisense nucleic acid molecule.

8. The method of claim 1, wherein the subject is also administered a pan-cancer phenotype therapeutic agent.

9-13. (canceled)

14. The method of claim 1, wherein the subject is RTEL1 reference or heterozygous for an RTEL1 variant nucleic acid molecule that comprises any one or more of the following genetic variations in the genomic nucleic acid molecule: 20:63687647:T:G, 20:63687688:C:G, 20:63693211:C:T, 20:63685789:A:C, 20:63662841:C:T, 20:63695551:CTG:C, 20:63667489:C:T, 20:63694876:C:T, 20:63661341:C:T, 20:63688315:G:A, 20:63662559:G:C, 20:63690137:A:C, 20:63667492:G:A, 20:63678268:T:C, 20:63690835:G:T, 20:63694489:G:A, 20:63687706:C:T, and 20:63667554:G:T, referring to the chromosome: positions set forth in the GRCh38/hg38 human genome assembly.

15. The method of claim 1, wherein the subject is RTEL1 reference or heterozygous for an RTEL1 variant nucleic acid molecule that comprises any one or more of the following genetic variations in the genomic nucleic acid molecule: 20:63667554:G:T, 20:63685789:A:C, 20:63693211:C:T (rs398123017), 20:63694489:G:A, and 20:63695551:CTG:C (rs1316931773), referring to the chromosome: positions set forth in the GRCh38/hg38 human genome assembly.

16. A method of treating a subject having a pan-cancer phenotype or at risk of developing a pan-cancer phenotype by administering a pan-cancer phenotype therapeutic agent, the method comprising:
determining or having determined whether the subject has a Regulator of Telomere Elongation Helicase 1 (RTEL1) variant nucleic acid molecule, by:
obtaining or having obtained a biological sample from the subject; and
performing or having performed a sequence analysis on the biological sample to determine if the subject has a genotype comprising an RTEL1 variant nucleic acid molecule; and
administering or continuing to administer the pan-cancer phenotype therapeutic agent in an amount that is the same as or less than a standard dosage amount, and/or an RTEL1 inhibitor to a subject that is RTEL1 reference;
administering or continuing to administer the pan-cancer phenotype therapeutic agent in an amount that is the same as or less than a standard dosage amount, and/or an RTEL1 inhibitor to a subject that is heterozygous for the RTEL1 variant nucleic acid molecule; or
administering or continuing to administer the pan-cancer phenotype therapeutic agent in a standard dosage amount to a subject that is homozygous for the RTEL1 variant nucleic acid molecule;
wherein the presence of the RTEL1 variant nucleic acid molecule indicates the subject has a decreased risk of developing pan-cancer phenotype; and
wherein the pan-cancer phenotype comprises adrenal cancer, bladder cancer, bone cancer, cartilage cancer, brain cancer, breast cancer, cervical cancer, colon cancer, esophagus cancer, kidney cancer, oral cavity cancer, pharynx cancer, liver cancer, lung cancer, melanoma, ovarian cancer, pancreatic cancer, prostate cancer, rectal cancer, stomach cancer, testicular cancer, thyroid cancer, or blood cancer.

17. (canceled)

18. The method of claim 16, wherein the RTEL1 inhibitor comprises an antisense nucleic acid molecule, a small interfering RNA (siRNA), and/or a short hairpin RNA (shRNA).

19. The method of claim 18, wherein the inhibitory nucleic acid molecule comprises an siRNA.

20. The method of claim 18, wherein the molecule comprises an antisense nucleic acid molecule.

21. The method of claim 16, wherein the method comprises administering or continuing to administer the pan-cancer phenotype therapeutic agent in an amount that is the same as or less than a standard dosage amount and the RTEL1 inhibitor to a subject that is heterozygous for the RTEL1 variant nucleic acid molecule.

22. The method of claim 16, wherein the method comprises administering or continuing to administer the pan-cancer phenotype therapeutic agent in an amount that is the same as or less than a standard dosage amount and the RTEL1 inhibitor to a subject that is RTEL1 reference.

23. The method of claim 16, wherein the RTEL1 variant nucleic acid molecule comprises a splice-site variant, a stop-gain variant, a start-loss variant, a stop-loss variant, a frameshift variant, a missense variant, an in-frame indel variant, and/or a variant that encodes a truncated RTEL1 variant polypeptide.

24. The method of claim 16, wherein the RTEL1 variant nucleic acid molecule comprises the genetic variation listed in Table 1, or an mRNA molecule produced therefrom, or a cDNA molecule produced from the mRNA molecule.

25. The method of claim 16, wherein the RTEL1 variant nucleic acid molecule comprises any one or more of the following genetic variations in the genomic nucleic acid molecule: 20:63687647:T:G, 20:63687688:C:G, 20:63693211:C:T, 20:63685789:A:C, 20:63662841:C:T, 20:63695551:CTG:C, 20:63667489:C:T, 20:63694876:C:T, 20:63661341:C:T, 20:63688315:G:A, 20:63662559:G:C, 20:63690137:A:C, 20:63667492:G:A, 20:63678268:T:C, 20:63690835:G:T, 20:63694489:G:A, 20:63687706:C:T, and 20:63667554:G:T, referring to the chromosome: positions set forth in the GRCh38/hg38 human genome assembly.

26. The method of claim 16, wherein the RTEL1 variant nucleic acid molecule comprises any one or more of the following genetic variations in the genomic nucleic acid molecule: 20:63667554:G:T, 20:63685789:A:C, 20:63693211:C:T (rs398123017), 20:63694489:G:A, and 20:63695551:CTG:C (rs1316931773), referring to the chromosome: positions set forth in the GRCh38/hg38 human genome assembly.

27-28. (canceled)

29. A method of identifying a subject having an increased risk of developing a pan-cancer phenotype, the method comprising:
determining or having determined the presence or absence of a Regulator of Telomere Elongation Helicase 1 (RTEL1) variant nucleic acid molecule in a biological sample obtained from the subject;
wherein:
when the subject is RTEL1 reference, then the subject has an increased risk of developing the pan-cancer phenotype; and
when the subject is heterozygous or homozygous for the RTEL1 variant nucleic acid molecule, then the subject has a decreased risk of developing the pan-cancer phenotype.

30-57. (canceled)
